(19) United States
(12) Patent Application Publication
(10) Pub. No.: US 2016/0082162 A1
(43) Pub. Date: Mar. 24, 2016

(54) Nanoparticle-Medicated Genetic Delivery of Growth Inhibiting Genes on Balloon Angioplasty to Suppress Intimal Hyperplasia

(71) Applicant: Board of Education of the Vocational Schools in the County of Bergen, Paramus, NJ (US)

(72) Inventors: Jenna Dirito, Saddle Brook, NJ (US); Serena Tharakan, Glen Rock, NJ (US); Robert Pergolizzi, New Milford, NJ (US)

(73) Assignee: Board of Education of the Vocational Schools in the County of Bergen, Paramus, NJ (US)

(21) Appl. No.: 14/704,037

(22) Filed: May 5, 2015

Related U.S. Application Data
(60) Provisional application No. 62/052,610, filed on Sep. 19, 2014.

(51) Int. Cl. A61L 31/16; A61M 25/10; A61L 29/16; A61L 31/10; A61L 29/08

(52) U.S. Cl. CPC A61L 31/16; A61L 31/10; A61L 29/085; A61L 29/16; A61M 25/104; A61L 2300/416; A61L 2300/258; A61M 2025/105; A61F 2/92

(57) ABSTRACT

The invention provides methods, devices, and reagents for treating a disease or a condition in a blood vessel, such as a venous or arterial disease or condition.

### CROSS-REFERENCE TO RELATED APPLICATIONS

The present application claims the priority of U.S. Provisional Application Ser. No. 62/052,610 filed Sep. 19, 2014, the disclosure of which is expressly incorporated herein by reference in its entirety.

### BACKGROUND OF THE INVENTION

Coronary artery disease constitutes a major cause of morbidity and mortality throughout the world, especially in the United States and Europe. Percutaneous transluminal coronary angioplasty (e.g., balloon angioplasty, with or without intracoronary stenting) is now a common and successful therapy for such disease. It is reported that more than 500,000 coronary bypass surgeries are performed annually in the United States alone. However, restenosis—the recurrence of an abnormal narrowing of an artery or valve after corrective surgery—occurs in as many as one-third to one-half of such revascularization procedures, usually within six months of the angioplasty procedure. For example, restenosis reportedly occurs at a rate of 25% to 50% in balloon angioplasties. The economic cost of restenosis has been estimated at $2 billion annually in the United States alone. Autopsy and atherectomy studies have identified intimal hyperplasia (IH) as the major histologic component of restenotic lesions. See Cerek et al., Am. J. Cardiol. 68:24C-33C, 1991.

Restenosis also remains a clinical concern in angioplasty that is performed in peripheral blood vessels. Likewise, stenosis is a clinical concern following transplantation of blood vessels (e.g., grafted veins and grafted artificial vessels) for cardiac bypass therapy or for treatment of peripheral ischemia or intermittent claudication, for example (e.g., above-knee, femoropopliteal arterial bypass grafts).

Mazur et al. (Texas Heart Institute Journal 21:104-111, 1994) state that restenosis is primarily a response of the artery to the injury caused by percutaneous coronary angioplasty, which disrupts the intimal layer of endothelial cells and underlying smooth muscle cells of the media. The authors state that multiple growth factors secreted by platelets, endothelial cells, macrophages, and smooth muscle cells are mechanistically involved in the restenosis process, and that proliferation of smooth muscle cells constitutes a critical pathogenetic feature. According to the authors, this smooth muscle cell proliferation has proven refractory to mechanical and pharmacologic therapy. More recently, others have called into question whether smooth muscle cell proliferation is of penultimate importance in restenosis. See Libby, Circ. Res. 82:404-406, 1998.

Chang & Leiden (Semin. Intervent. Cardiol. 1:185-193, 1996), incorporated herein by reference, review somatic gene therapy approaches to treat restenosis. Chang and Leiden suggest that replication-deficient adenoviruses comprise a promising and safe vector system for gene therapy directed toward prevention of restenosis, because such viruses can efficiently infect a wide variety of cell types, including vascular smooth muscle cells; such viruses can be produced at high titers (e.g., 10¹⁰-10¹² plaque forming units per milliliter); such viruses can accommodate a transgene insert of, e.g., 7-9 kilobases (kb) in size; such viruses can be delivered percutaneously through standard catheters; and such viruses do not integrate into the host genome. Chang & Leiden also reviewed cytotoxic and cytostatic gene therapy approaches, designed to kill or arrest proliferating vascular smooth muscle cells thought to be responsible for neointimal formations that characterize restenosis.

The role played by the Vascular Endothelial Growth Factor (VEGF) gene in intimal hyperplasia has been an area of certain controversy. While opposite views exists, Isner & Asahara (WO 98/19712, incorporated herein by reference) teach treating injured blood vessels and accelerating reendothelialization following angioplasty by isolating a patient's endothelial progenitor cells and re-administering such cells to the patient, and suggest that the effectiveness of using an angiogenesis-promoting growth factor, such as vascular endothelial growth factor (VEGF) or basic fibroblast growth factor (bFGF), may be limited by the lack of endothelial cells on which the VEGF or bFGF will exert its effect. Likewise, DeYoung & Dichek (Circ. Res. 82:306-313, 1998) state that VEGF gene delivery does not currently appear destined for application to human coronary restenosis, and that two independent studies suggest that VEGF delivery may actually worsen arterial intimal hyperplasia.

On the other hand, pancreatic and duodenal homeobox 1 (Pdx1) is a gene that has generally been studied regarding its role in diabetes. The relationship between Pdx1 and VEGF has been poorly investigated, especially in regards to intimal hyperplasia.

The development of an aggressive treatment for intimal hyperplasia will ultimately save a countless number of lives and drastically reduce patient expense and discomfort, as fewer surgeries will be needed once the cycle of re-stenosis is broken. Yet there is still a long-felt need for improvements to angioplasty materials and/or methods, and/or for adjunct therapies, to reduce instances of restenosis.

### SUMMARY OF THE INVENTION

The invention described herein addresses long-felt needs in the field of medicine by providing materials and methods for the prevention of stenosis or restenosis in mammalian blood vessels. It helps to end the necessity for repetitive detrimental balloon angioplasty and stent placement procedures, thus saves time, energy, and money. Without the need for multiple surgeries, patients have more sustainable and less susceptible immune systems, thus increasing the general health of those in need of the described procedures.

Thus one aspect of the invention provides a medical device comprising a surface designed to contact the lumen of a blood vessel in a mammal, wherein the surface is coated by a composition comprising a VEGF-A inhibitor and/or a Pdx1 inhibitor, and wherein said VEGF-A inhibitor inhibits expression of VEGF-A, and said Pdx1 inhibitor inhibits expression of Pdx1.

In certain embodiments, the composition further comprises glycerol.

In certain embodiments, the medical device is a stent (e.g., an intravascular stent), a catheter (e.g., an intravascular catheter, a balloon catheter), an angioplastic balloon, an extravascular collar, an elastomeric membrane adapted to cover a surface of an intravascular stent or catheter, or a combination thereof.

Another aspect of the invention provides a method of treating or preventing stenosis, restenosis, or intimal hyperplasia (IH) in a mammal in need of treatment or prevention, the method comprising administering a therapeutically or prophylactically effective amount of a Pdx1 inhibitor and/or a VEGF-A inhibitor to the mammal in need thereof.

In certain embodiments, the Pdx1 inhibitor and/or the VEGF-A inhibitor is administered by contacting the lumen of a blood vessel in the mammal afflicted with stenosis, restenosis, or intimal hyperplasia (IH) with a surface of a medical device, wherein the surface is coated by a composition comprising the VEGF-A inhibitor and/or the Pdx1 inhibitor.

In certain embodiments, the mammal is a human, or a rodent (e.g., a rat).

In certain embodiments, the VEGF-A inhibitor and/or the Pdx1 inhibitor is administered prophylactically to the blood vessel shortly before, concurrently with, or shortly after an angioplasty procedure, or a procedure to perform a vascular graft.

In certain embodiments, the VEGF-A inhibitor and/or the Pdx1 inhibitor is administered with a device employed in the angioplasty selected from the group consisting of a catheter, a stent, an expandable elastic membrane, and a combination thereof.

In certain embodiments, the VEGF-A inhibitor and/or the Pdx1 inhibitor is administered with a device used in a vascular graft procedure (e.g., an extravascular collar).

In certain embodiments, the blood vessel is an artery (or a vein).

In certain embodiments, the Pdx1 inhibitor and/or the VEGF-A inhibitor is a polynucleotide.

In certain embodiments, the polynucleotide inhibits Pdx1 expression and/or VEGF-A expression via RNA interference (RNAi).

In certain embodiments, the polynucleotide is an shRNA (short hairpin RNA), a dsRNA that can be processed by an RNAse III into siRNA, or an miRNA or precursor thereof.

In certain embodiments, the polynucleotide comprises a modified sugar moiety (e.g., 2-O-Me), a modified base moiety (e.g., nebularine or xanthosine nucleotide), a modified inter-sugar linkage (e.g., phosphorothioate), or combinations thereof.

In certain embodiments, the polynucleotide comprises a locked nucleic acid (LNA™), a deoxyribonucleic acid (DNA), a ribonucleic acid (RNA), or a combination thereof.

In certain embodiments, the polynucleotide encodes a product that inhibits Pdx1 expression and/or VEGF-A expression via RNA interference (RNAi).

In certain embodiments, the product is an shRNA (short hairpin RNA), a dsRNA that can be processed by an RNAse III into siRNA, or an miRNA or precursor thereof.

In certain embodiments, the product is expressed from an operably linked promoter on the polynucleotide.

In certain embodiments, expression of the product in endothelial cells of the blood vessel contacted by the surface leads to reduced expression of Pdx1 and/or VEGF-A in said endothelial cells.

In certain embodiments, expression of the product in endothelial cells of the blood vessel contacted by the surface leads to inhibition of stenosis, restenosis, or IH of the blood vessel.

In certain embodiments, the polynucleotide is a plasmid vector (e.g., naked DNA plasmid vector), or a viral vector (e.g., adenoviral vector preferably a replication-deficient adenoviral vector, AAV vector, retroviral vector, lentiviral vector, lipofectin-mediated gene transfer vector, liposome).

In certain embodiments, the method further comprises identifying the mammal in need of treatment as being a candidate for administering the Pdx1 inhibitor and/or the VEGF-A inhibitor.

In certain embodiments, the mammal has been treated for a stenosed blood vessel, has a stenosed blood vessel, or will be treated for a stenosed blood vessel.

In certain embodiments, the blood vessel is a grafted blood vessel.

Additional features and variations of the invention will be apparent to those skilled in the art from the entirety of this application, and all such features are intended as aspects of the invention.

Likewise, any feature or embodiment of the invention described herein can be combined with any other embodiments into additional embodiments that also are intended as aspects of the invention, irrespective of whether the combination of features is specifically mentioned above as one aspect of the invention.

### BRIEF DESCRIPTION OF THE DRAWINGS

FIG. 1 shows the gene map for the anti-Pdx1 plasmid. The U6 promoter drives the expression of an shRNA that inhibits the expression of the target Pdx1 gene. The sequences for forming the 29-bp stem and 7-nucleotide loop for the shRNA are shown. Expression of the GFP coding sequence is driven by the CMV promoter on the same plasmid.

FIG. 2A shows a cross-section of a left carotid artery (100×) harvested from a control rat having received a standard carotid artery injury. The arrow points to an area of the vessel having intimal regrowth, or intimal hyperplasia (IH).

FIG. 2B shows a cross-section of a left carotid artery (100×) harvested from a control rat that was inflated with a glycerol-coated balloon, without nanoparticles containing shRNA encoding plasmid. The arrow points to portions of the vessel having intimal regrowth, or intimal hyperplasia. As expected, this control artery also displays high levels of intimal hyperplasia.

FIG. 2C shows a cross-section of a left carotid artery harvested from an experimental group rat that was inflated with the glycerol-nanoparticle solution-coated balloon, which contained an shRNA-encoding plasmid. This artery displays very little presence of intimal hyperplasia, supporting the idea that the gene therapy was in fact delivered to the artery, thereby reducing the level of intimal hyperplasia.

FIG. 3 is a TEM image of PLGA nanoparticles containing the anti-Pdx1 shRNA-encoding plasmid. The nanoparticles are roughly 200 nm in size, and appear properly formed, confirming the quality of the preparation.

FIG. 4 shows the levels of intimal hyperplasia among the simple control group (group 1), the glycerol-only coated balloon control group (group 2), and the glycerol-nanoparticle experimental group (group 3). While the difference between group 1 and group 2 is insignificant (p-value of 0.959), the difference between group 2 and group 3 is statistically significant (p-value of 1.9e-14). This suggests that glycerol alone has no significant effect on the levels of intimal hyperplasia, but glycerol containing nanoparticles packaged with the shRNA plasmid did in fact lower the levels of intimal hyperplasia observed.

FIG. 5 shows the relative quantification of VEGF-A as determined by rt-PCR. The glycerol-control condition, the tall left bar, and the experimental condition, the short right bar, was compared to a standard, which was then used to determine relative quantification. VEGF-A levels were significantly lowered (p-value of 6.29e-60) in the experimental condition as compared to the glycerol-control.

### DETAILED DESCRIPTION OF THE INVENTION

### 1. Overview

The present invention is based on the discovery that Pdx1 appears to be an upstream stimulator of VEGF-A expression, and when expression of Pdx1 and/or VEGF-A is inhibited, e.g., when an inhibitor for Pdx1 or VEGF-A is administered to a mammal that has suffered a vascular trauma, such as the trauma that can occur during conventional balloon angioplasty procedures, restenosis of the injured vessel is reduced or eliminated.

According to the invention described herein, Pdx1 is a gene which may play a key role in regulating the expression of VEGF-A, both of which may allow for the growth of the intima, and therefore the development of IH. By inhibiting the expression of one or both genes, IH may be hindered or prevented in patients in need thereof.

In one of the exemplary embodiments, expression of Pdx1 and/or VEGF-A is inhibited in endothelial cells in blood vessel (e.g., an artery), by using RNA interference (RNAi) induced by nanoparticle-mediated delivery of shRNA via a surgical balloon coated with a nanoparticle-glycerol suspension. This allowed for direct delivery of an inhibitor of a target gene during a carotid artery injury model surgery performed on rats. During this surgery, the balloon construct was inserted into the artery, thereby distributing the glycerol nanoparticle solution (and therefore the shRNA inhibitors of the target gene(s)) to the walls of the artery. The images obtained from harvested tissue showed that the control arteries—those that did not receive the nanoparticle treatment but were otherwise identically treated—developed significant levels of IH. However, through histology, it was observed that the experimental arteries, those that had Pdx1 expression knockdown via the nanoparticles, showed little presence of IH. This suggests that the method of gene delivery was effective. Nanoparticle-mediated gene delivery to the artery was further verified as the nanoparticles contained a green fluorescent protein (GFP) gene that was visible by fluorescence microscopy. Finally, reverse transcriptase PCR (RT-PCR) was used to detect the levels of VEGF-A in the cell. The PCR confirmed that the experimental condition had resulted in much lower levels of VEGF-A.

Thus one aspect of the invention provides a medical device comprising a surface designed to contact the lumen of a blood vessel in a mammal, wherein the surface is coated by a composition comprising a VEGF-A inhibitor and/or a Pdx1 inhibitor, and wherein said VEGF-A inhibitor inhibits expression of VEGF-A, and said Pdx1 inhibitor inhibits expression of Pdx1.

In certain embodiments, the composition further comprises glycerol, which may be present in the formulation of the subject Pdx1/VEGF-A inhibitors that coats the medical device.

In certain embodiments, the medical device is a stent (e.g., an intravascular stent), a catheter (e.g., an intravascular catheter, a balloon catheter), an angioplastic balloon, an extravascular collar, an elastomeric membrane adapted to cover a surface of an intravascular stent or catheter, or a combination thereof.

Another aspect of the invention provides a method of treating or preventing stenosis, restenosis, or intimal hyperplasia (IH) in a mammal in need of treatment or prevention, the method comprising administering a therapeutically or prophylactically effective amount of a Pdx1 inhibitor and/or a VEGF-A inhibitor to the mammal in need thereof.

In certain embodiments, the Pdx1 inhibitor and/or the VEGF-A inhibitor is administered by contacting the lumen of a blood vessel in the mammal afflicted with stenosis, restenosis, or intimal hyperplasia (IH) with a surface of a medical device, wherein the surface is coated by a composition comprising the VEGF-A inhibitor and/or the Pdx1 inhibitor.

In certain embodiments, the mammal is a human, a non-human primate, a non-human mammal, a livestock animal (e.g., cattle, horse, pig, sheep, goat), a pet (e.g., a cat, a dog), an experimental/model animal, a rodent (e.g., mouse, rat, hamster, rabbit), or a marine mammal.

In certain embodiments, the VEGF-A inhibitor and/or the Pdx1 inhibitor is administered prophylactically to the blood vessel shortly before (e.g., several months, weeks, days, hours, or minutes before), concurrently with, or shortly after (e.g., several months, weeks, days, hours, or minutes after) an angioplasty procedure, or a procedure to perform a vascular graft.

In certain embodiments, the VEGF-A inhibitor and/or the Pdx1 inhibitor is administered with a device employed in the angioplasty selected from the group consisting of a catheter, a stent, an expandable elastic membrane, and a combination thereof.

In certain embodiments, the VEGF-A inhibitor and/or the Pdx1 inhibitor is administered with a device used in a vascular graft procedure (e.g., an extravascular collar).

In certain embodiments, the blood vessel is an artery (or a vein).

In certain embodiments, the Pdx1 inhibitor and/or the VEGF-A inhibitor is a polynucleotide.

In certain embodiments, the polynucleotide inhibits Pdx1 expression and/or VEGF-A expression via RNA interference (RNAi), CRISPR/Cas or TALEN or ZFN mediated silencing.

In certain embodiments, the polynucleotide is an shRNA (short hairpin RNA), a dsRNA that can be processed by an RNAse III into siRNA, or an miRNA or precursor thereof.

In certain embodiments, the polynucleotide comprises a modified sugar moiety (e.g., 2-O-Me), a modified base moiety (e.g., nebularine or xanthosine nucleotide), a modified inter-sugar linkage (e.g., phosphorothioate), or combinations thereof.

In certain embodiments, the polynucleotide comprises a locked nucleic acid (LNA™), a deoxyribonucleic acid (DNA), a ribonucleic acid (RNA), or a combination thereof.

In certain embodiments, the polynucleotide encodes a product that inhibits Pdx1 expression and/or VEGF-A expression via RNA interference (RNAi), CRISPR/Cas or TALEN or ZFN mediated silencing.

In certain embodiments, the product is an shRNA (short hairpin RNA), a dsRNA that can be processed by an RNAse III into siRNA, or an miRNA or precursor thereof.

In certain embodiments, the product is expressed from an operably linked promoter on the polynucleotide. The promoter may be operable in a mammalian cell. Preferably, the promoter is selectively operable in a cell of the blood vessel, such as an endothelial cell. In certain embodiments, the promoter does not direct transcription of its downstream gene product in a cell that is not in the blood vessel wall (e.g., non-endothelial cells).

In certain embodiments, expression of the product in endothelial cells of the blood vessel contacted by the surface leads to reduced expression of Pdx1 and/or VEGF-A in said endothelial cells. For example, the reduction is at least about 20%, 30%, 40%, 50%, 60%, 70%, 80%, 90%, 95%, 97%, 99% or more compared to a vector control.

In certain embodiments, expression of the product in endothelial cells of the blood vessel contacted by the surface leads to inhibition of stenosis, restenosis, or IH of the blood vessel. Intima/media (I/M) ratio can be used as a parameter for measuring intimal thickening and reduction thereof.

In certain embodiments, the polynucleotide is a plasmid vector (e.g., naked DNA plasmid vector), or a viral vector (e.g., adenoviral vector preferably a replication-deficient adenoviral vector, AAV vector, retroviral vector, lentiviral vector, lipofectin-mediated gene transfer vector, liposome).

In certain embodiments, the method further comprises identifying the mammal in need of treatment as being a candidate for administering the Pdx1 inhibitor and/or the VEGF-A inhibitor.

In certain embodiments, the mammal has been treated for a stenosed blood vessel, has a stenosed blood vessel, or will be treated for a stenosed blood vessel.

In certain embodiments, the blood vessel is a grafted blood vessel.

Yet another aspect of the invention provides a method of treating or preventing a disease or condition in a mammal in need of treatment or prevention, the method comprising administering a therapeutically or prophylactically effective amount of a Pdx1 inhibitor and/or a VEGF-A inhibitor to the mammal in need thereof, wherein the disease or condition is: breast cancer (e.g., VEGF-overexpressing breast cancer), rheumatoid arthritis (RA), diabetic retinopathy (DR), the wet form age-related macular degeneration (AMD), angiosarcoma, undesirable angiogenesis (such as angiogenesis in cancer or solid tumor), a kidney disease associated with VEGF overexpression (such as glomerular hypertrophy or proteinuria), or pre-eclampsia.

In certain embodiments, the method further comprising administering a second therapeutic agent effective to treat the disease or condition. For example, the second therapeutic agent may be effective against VEGF-overexpressing (breast) cancer, such as bevacizumab (Avastin), antibody derivatives such as ranibizumab (Lucentis), orally-available small molecules that inhibit the tyrosine kinases stimulated by VEGF such as lapatinib (Tykerb), sunitinib (Sutent), sorafenib (Nexavar), axitinib, AZ2171 (Cediranib), pazopanib, and thiazolidinediones. Some of these therapies target VEGF receptors rather than the VEGFs. The second therapeutic agent effective to treat the disease or condition may also include THC and Cannabidiol for slowing Glioma growth.

With the invention generally described above, certain features of the invention are further described in the sections below.

### 2. Pdx1 Inhibitors and VEGF-A Inhibitors

In certain embodiments, the Pdx1/VEGF-A inhibitors of the invention may be a polynucleotide that encodes a product that inhibits expression of Pdx1 and/or VEGF-A in a host cell, such as an antisense RNA, a ribozyme, an shRNA, an siRNA or miRNA or precursor thereof that can be processed by an RNAse III (such as Dicer and/or Drosha), or a guide sequence for gene targeting based on a CRISPR/Cas system (e.g., CRISPR/Type II Cas-9 system), zinc-finger nucleases (ZFNs), or TALEN (transcription activator-like effector nucleases), each of which may inhibit the expression of a host Pdx1 and/or VEGF-A gene.

For example, concerning the CRISPR/Cas system, the polynucleotide may encode a type II Cas-9 protein and a CRISPR/Cas-9 guide sequence designed to target Pdx1 and/or VEGF-A.

In certain embodiments, the product may be transcribed from an operably linked promoter on the polynucleotide.

The polynucleotide of the invention may be recombinant expression vectors comprising recombinant nucleic acids (e.g., “products”) operatively linked to an expression control sequence, wherein expression, i.e., the transcription and optionally further processing, results in one or more polynucleotide or product of the invention or a precursor molecules thereof.

The vector may be a DNA vector, e.g., a viral vector or plasmid, particularly an expression vector suitable for nucleic acid expression in eukaryotic, more particularly mammalian cells. The recombinant nucleic acid contained in the vector may be a sequence which results in the transcription of the oligonucleotide of the invention as such, a precursor or primary transcript thereof, which may be further processed to give the oligonucleotide of the invention.

In certain embodiments, the vector may be administered in vivo (e.g., through a coated medical device for expression in endothelial cells of the blood vessel) to thereby initiate therapeutic or prophylactic treatment, by expression of one or more copies of the polynucleotide or product of the invention. In certain embodiments, use of vectors may be advantageous because the vectors can be more stable than polynucleotides and thus effect long-term expression of the polynucleotide/product of the invention.

Vectors may be designed for delivery of multiple polynucleotides of the invention capable of antagonizing multiple targets (e.g., Pdx1 and VEGF-A). Accordingly, in one embodiment, a vector is contemplated that expresses a plurality of the polynucleotide/product of the invention.

In one embodiment, the vector encodes about 2, 3, 4 or more polynucleotide/product of the invention, each of which may target the same or different target.

In one embodiment, expression of the polynucleotide of the invention is driven by a RNA polymerase III (pol III) promoter (T. R. Brummelkamp et al., Science, (2002) 296:550-553; P J. Paddison et al., Genes Dev., (2002) 16:948-958). Pol III promoters are advantageous because their transcripts are not necessarily post-transcriptionally modified, and because they are highly active when introduced in mammalian cells. In another embodiment, expression of the oligonucleotide of the invention is driven by a RNA polymerase II (pol II) promoter. Polymerase II (pol II) promoters may offer advantages to pol III promoters, including being more easily incorporated into viral expression vectors, such as retroviral and adeno-associated viral vectors, and the existence of inducible and tissue specific pol II dependent promoters.

In a related embodiment, the Pdx1/VEGF-A inhibitors of the invention may be the product itself, e.g., the antisense RNA, ribozyme, shRNA, siRNA or miRNA or precursor thereof that can be processed by an RNAse III (such as Dicer and/or Drosha), each of which may inhibit the expression of a host Pdx1 and/or VEGF-A gene.

In this embodiment, the polynucleotide may contain one or more modified nucleotides (see section below) that may further enhance the bioavailability, solubility, stability, and/or uptake (by host cell) of the modified polynucleotide.

In an exemplary embodiment, the Pdx1 inhibitor inhibits Pdx1 expression via RNAi mechanism. In an exemplary embodiment, the VEGF-A inhibitor inhibits VEGF-A expression via RNAi mechanism.

The inhibitor may be an shRNA, an siRNA or miRNA or precursor thereof that can be processed by an RNAse III (such as Dicer and/or Drosha), each of which may be designed based on the sequence of the host Pdx1 gene, using standard molecular biology methods or commercial sources (see Silencer® Select siRNAs from Ambion/Applied Biosys). Once the RNAi based inhibitor has been designed, there are several methods for preparing the siRNA, such as chemical synthesis, in vitro transcription, siRNA expression vectors, and PCR expression cassettes. Each may be used in the methods and devices of the invention.

Irrespective of which method one uses, the first step in designing a siRNA is to choose the siRNA target site. The exemplary guidelines described below can help to choose siRNA target sites with approximately half of all siRNAs yield >50% reduction in target mRNA levels.

Specifically, siRNA target sites may be chosen in a variety of different organisms based on the following guidelines. First, a target mRNA sequence about 21-nt in length and beginning with an AA dinucleotide is selected. Each AA and its 3′ adjacent 19 nucleotides constitute a potential siRNA target site. Next, 2-4 such potential target sites are selected. Typically, more than half of randomly designed siRNAs provide at least a 50% reduction in target mRNA levels and approximately 1 of 4 siRNAs provide a 75-95% reduction. Choose target sites from among the sequences identified in the first step based on the following guidelines: (1) siRNAs with 30-50% GC content are more active than those with a higher G/C content; (2) since a 4-6 nucleotide poly(T) tract acts as a termination signal for RNA pol III, avoid stretches of >4 T's or A's in the target sequence when designing sequences to be expressed from an RNA pol III promoter; (3) since some regions of mRNA may be either highly structured or bound by regulatory proteins, siRNA target sites at different positions are selected along the length of the gene sequence; and (4) compare the potential target sites to the appropriate genome database (human, mouse, rat, etc.) and eliminate from consideration any target sequences with more than 16-17 contiguous base pairs of homology to other coding sequences. NCBI BLAST can be used for this purpose.

Last but not least, appropriate controls should be designed. A complete siRNA experiment should include a number of controls to ensure the validity of the data. Exemplary controls include: (a) a negative control siRNA with the same nucleotide composition as the chosen siRNA but which lacks significant sequence homology to the genome. To design such a negative control siRNA, scramble the nucleotide sequence of the gene-specific siRNA and conduct a search to make sure it lacks homology to any other gene; (b) additional siRNA sequences targeting the same mRNA. Perhaps the best way to ensure confidence in RNAi data is to perform experiments, using a single siRNA at a time, with two or more different siRNAs targeting the same gene. Prior to these experiments, each siRNA should be tested to ensure that it reduces target gene expression by comparable levels.

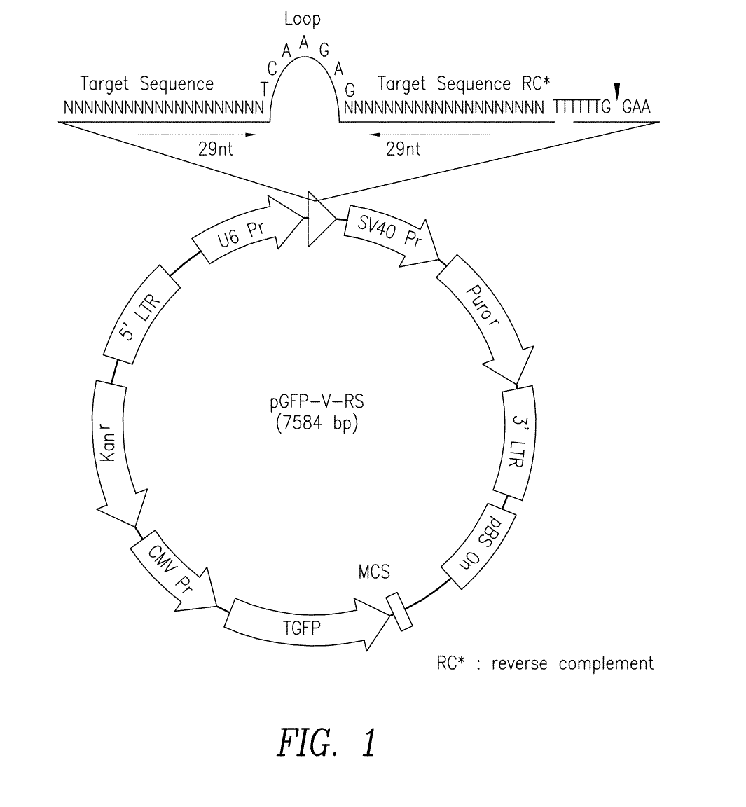

Specific guidelines also apply for designing siRNA hairpins (shRNA) encoded by siRNA expression vectors and siRNA expression cassettes. Most of the designs have two inverted repeats separated by a short spacer sequence and ended with a string of T's that served as a transcription termination site. These designs produce an RNA transcript that is predicted to fold into a short hairpin siRNA (see FIG. 1). The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can all be adjusted.

In an exemplary embodiment, siRNA hairpin design include a first step of designing an appropriate insert, based on the general guideline for choosing siRNA target sites described above. For screening, four siRNA sequences per target are tested, spacing the siRNA sequences down the length of the gene sequence to reduce the chances of targeting a region of the mRNA that is either highly structured or bound by regulatory proteins. PCR-derived siRNA expression cassettes (SECs), which are PCR products that include promoter and terminator sequences flanking a hairpin siRNA template, may be used to facilitate this process. This screening strategy also permits the rapid identification of the best combination of promoter and siRNA sequence in the experimental system. SECs found to effectively elicit gene silencing can be readily cloned into a vector for long term studies. Sequences that function well as transfected siRNAs usually also function well as siRNAs that are expressed in vivo. The only exception is that siRNA sequences to be expressed in vivo should not contain a run of 4 or 5 A's or T's, as these can act as termination sites for Polymerase III.

For traditional cloning into pSilencer vectors, two DNA oligonucleotides that encode the chosen siRNA sequence are designed for insertion into the vector. In general, the DNA oligonucleotides consist of a 19-nucleotide sense siRNA sequence linked to its reverse complementary antisense siRNA sequence by a short spacer. A 9-nucleotide spacer (TTCAAGAGA) may be used, although other spacers can be designed as well. 5-6 T's can be added to the 3′ end of the oligonucleotide. In addition, for cloning into certain vectors, suitable restriction endonuclease sites (e.g., nucleotide overhangs to the EcoR I restriction site) can be added to the 5′ and 3′ end of the DNA oligonucleotides. The resulting RNA transcript is expected to fold back and form a stem-loop structure comprising a 19 bp stem and a (9 nt) loop with 2-3 U's at the 3′ end.

For cloning into the CMV-based vector system, one notable exception is the absence of 5-6 T's from the 3′-end of the oligonucleotides for such vector system since the transcription termination signal for the CMV-based vector system is provided by the SV40 polyA terminator.

A hairpin siRNA expression cassette is usually constructed to contain the sense strand of the target, followed by a short spacer, then the antisense strand of the target, in that order. Alternatively, reversal of the order of sense and antisense strands within the siRNA expression constructs may be used.

There appears to be some degree of variation in the length of nucleotide sequence being used as the stem of siRNA expression cassette. In certain embodiments, about 19 nucleotides-long sequences are used as the stem of siRNA expression cassette. In contrast, other siRNA stems ranging from 21 nucleotides-long to 25-29 nucleotides-long may be used. It is found that hairpin siRNAs with these various stem lengths all function well in gene silencing studies.

Successful gene silencing results have been obtained using hairpin siRNAs with loop size ranging between 3 to 23 nucleotides (e.g., 3, 4, 5, 6, 7, 9, and 23). Any of these can be used as loop size.

In certain embodiments, a 5′ overhang is present in designed shRNA construct. For example, a 6 nucleotide 5′ overhang may be included in the hairpin siRNA construct. Hairpin siRNAs with 5′ overhangs have been shown to be functional in gene silencing.

Ohlsson et al. (EMBO J. 12:4251-4259, 1993) cloned mouse Pdx1 from an insulinoma cell line. The deduced 284-amino acid protein has a central homeodomain and a calculated molecular mass of 31 kD. Northern blot analysis of several mouse tissues and cells lines detected Pdx1 only in the mouse insulinoma cell line and in insulin-producing cell lines from other species.

The human Pdx1 nucleotide sequence is reproduced below based on NCBI RefSeq NM_{—}000209, which sequence may be used as query to identify any other mammalian Pdx1 sequences, and for designing antisense, ribozyme, RNAi (including shRNA), or CRISPR/Cas based inhibitor constructs:

| | |
|---|---|
| 1 | gggtggcgcc gggagtggga acgccacaca gtgccaaatc cccggctcca gctcccgact |
| 61 | cccggctccc ggctcccggc tcccggtgcc caatcccggg ccgcagccat gaacggcgag |
| 121 | gagcagtact acgcggccac gcagctttac aaggacccat gcgcgttcca gcgaggcccg |
| 181 | gcgccggagt tcagcgccag cccccctgcg tgcctgtaca tgggccgcca gcccccgccg |
| 241 | ccgccgccgc acccgttccc tggcgccctg ggcgcgctgg agcagggcag ccccccggac |
| 301 | atctccccgt acgaggtgcc ccccctcgcc gacgaccccg cggtggcgca ccttcaccac |
| 361 | cacctcccgg ctcagctcgc gctcccccac ccgcccgccg ggcccttccc ggagggagcc |
| 421 | gagccgggcg tcctggagga gcccaaccgc gtccagctgc ctttcccatg gatgaagtct |
| 481 | accaaagctc acgcgtggaa aggccagtgg gcaggcggcg cctacgctgc ggagccggag |
| 541 | gagaacaagc ggacgcgcac ggcctacacg cgcgcacagc tgctagagct ggagaaggag |
| 601 | ttcctattca acaagtacat ctcacggccg cgccgggtgg agctggctgt catgttgaac |
| 661 | ttgaccgaga gacacatcaa gatctggttc caaaaccgcc gcatgaagtg gaaaaaggag |
| 721 | gaggacaaga agcgcggcgg cgggacagct gtcgggggtg gcggggtcgc ggagcctgag |
| 781 | caggactgcg ccgtgacctc cggcgaggag cttctggcgc tgccgccgcc gccgcccccc |
| 841 | ggaggtgctg tgccgcccgc tgcccccgtt gccgcccgag agggccgcct gccgcctggc |
| 901 | cttagcgcgt cgccacagcc ctccagcgtc gcgcctcggc ggccgcagga accacgatga |
| 961 | gaggcaggag ctgctcctgg ctgaggggct tcaaccactc gccgaggagg agcagagggc |
| 1021 | ctaggaggac cccgggcgtg gaccacccgc cctggcagtt gaatggggcg gcaattgcgg |
| 1081 | ggcccacctt agaccgaagg ggaaaacccg ctctctcagg cgcatgtgcc agttggggcc |
| 1141 | ccgcgggtag atgccggcag gccttccgga agaaaaagag ccattggttt ttgtagtatt |
| 1201 | ggggccctct tttagtgata ctggattggc gttgtttgtg gctgttgcgc acatccctgc |
| 1261 | cctcctacag cactccacct tgggacctgt ttagagaagc cggctcttca aagacaatgg |
| 1321 | aaactgtacc atacacattg gaaggctccc taacacacac agcggggaag ctgggccgag |
| 1381 | taccttaatc tgccataaag ccattcttac tcgggcgacc cctttaagtt tagaaataat |
| 1441 | tgaaaggaaa tgtttgagtt ttcaaagatc ccgtgaaatt gatgccagtg gaatacagtg |
| 1501 | agtcctcctc ttcctcctcc tcctcttccc cctccccttc ctcctcctcc tcttcttttc |
| 1561 | cctcctcttc ctcttcctcc tgctctcctt tcctccccct cctcttttcc ctcctcttcc |
| 1621 | tcttcctcct gctctccttt cctccccctc ctctttctcc tcctcctcct cttcttcccc |
| 1681 | ctcctctccc tcctcctctt cttccccctc ctctccctcc tcctcttctt ctccctcctc |
| 1741 | ttcctcttcc tcctcttcca cgtgctctcc tttcctcccc ctcctcttgc tccccttctt |
| 1801 | ccccgtcctc ttcctcctcc tcctcttctt ctccctcctc ttcctcctcc tctttcttcc |
| 1861 | tgacctcttt ctttctcctc ctcctccttc tacctcccct tctcatccct cctcttcctc |
| 1921 | ttctctagct gcacacttca ctactgcaca tcttataact tgcacccctt tcttctgagg |
| 1981 | aagagaacat cttgcaaggc agggcgagca gcggcagggc tggcttagga gcagtgcaag |
| 2041 | agtccctgtg ctccagttcc acactgctgg cagggaaggc aaggggggac gggcctggat |
| 2101 | ctgggggtga gggagaaaga tggacccctg ggtgaccact aaaccaaaga tattcggaac |
| 2161 | tttctattta ggatgtggac gtaattcctg ttccgaggta gaggctgtgc tgaagacaag |
| 2221 | cacagtggcc tggtgcgcct tggaaaccaa caactattca cgagccagta tgaccttcac |
| 2281 | atctttagaa attatgaaaa cgtatgtgat tggagggttt ggaaaaccag ttatcttatt |
| 2341 | taacatttta aaaattacct aacagttatt tacaaacagg tctgtgcatc ccaggtctgt |
| 2401 | cttcttttca aggtctgggc cttgtgctcg ggttatgttt gtgggaaatg cttaataaat |
| 2461 | actgataata tgggaagaga tgaaaactga ttctcctcac tttgtttcaa acctttctgg |
| 2521 | cagtgggatg attcgaattc acttttaaaa ttaaattagc gtgttttgtt ttg |

For example, using this sequence as query for the NCBI BLASTn program, at least the following mammalian Pdx1 sequences have been identified, each can be used to design the Pdx1 inhibitors of the invention: XM_{—}004054304.1 for Gorilla gorilla gorilla (western lowland gorilla); XM_{—}002824122.2 for Pongo abelii (Sumatran orangutan); XM_{—}003270212.2 for Nomascus leucogenys (northern white-cheeked gibbon); XM_{—}008021806.1 for Chlorocebus sabaeus (green monkey); XM_{—}005585535.1 for Macaca fascicularis (crab-eating macaque); XM_{—}003913711.1 for Papio anubis (olive baboon); NM_{—}001081478.1 for Pan troglodytes (chimpanzee); XM_{—}002748914.1 for Callithrix jacchus (white-tufted-ear marmoset); NM_{—}001141984.1 for Sus scrofa (pig); NM_{—}001192136.1 for Bos taurus (cattle); XM_{—}001492067.1 for Equus caballus (horse); NM_{—}001284471.1 for Canis lupus familiaris (dog); XM_{—}006927207.1 for Felis catus (domestic cat); XM_{—}004281767.1 for Orcinus orca (killer whale); XM_{—}006748898.1 for Leptonychotes weddellii (Weddell seal); and NM_{—}008814.3 for Mus musculus (house mouse).

Thus in certain embodiments, the mammal is a human, a non-human primate, a non-human mammal, a livestock animal (e.g., cattle, horse, pig, sheep, goat), a pet (e.g., a cat, a dog), an experimental/model animal, a rodent (e.g., mouse, rat, hamster, rabbit), or a marine mammal.

Multiple human VEGF-A transcripts exist, including NM_{—}001171623 (transcript variant 1); NM_{—}001171624.1 (transcript variant 2); NM_{—}001171625.1 (transcript variant 3); NM_{—}001171626.1 (transcript variant 4); NM_{—}001171627.1 (transcript variant 5); NM_{—}001171628.1 (transcript variant 6); NM_{—}001171629.1 (transcript variant 7); and NM_{—}001171630.1 (transcript variant 8), each of which may be used as query to identify additional mammalian VEGF sequences for designing the various VEGF-A inhibitors.

### 3. Modified Nucleotides

In certain embodiments, the polynucleotide of the invention may comprise one or more modified nucleotides or chemical modifications to, for example, enhance a desired property, such as to enhance its stability (e.g., to prevent degradation), to promote its cellular uptake, to enhance targeting efficiency and/or affinity to its binding partner, to improve patient tolerance, and/or to reduce toxicity, etc.

As used herein, “nucleoside” includes the unit made up of a heterocyclic base and its sugar. “Nucleotide” includes a nucleoside having a phosphate group on its 3′ or 5′ sugar hydroxyl group. “Oligonucleotide” or “polynucleotide,” which may be used interchangeably herein, includes a plurality of joined nucleotide units formed in a specific sequence from naturally occurring bases and pentofuranosyl groups joined through a sugar group by native phosphodiester bonds. These nucleotide units may be nucleic acid bases such as guanine (G), adenine (A), cytosine (C), thymine (T), or uracil (U). The sugar group may be a deoxyribose or ribose. This term also includes both naturally occurring and synthetic species formed from naturally occurring subunits.

In certain embodiments, polynucleotide may also include “polynucleotide analogue” that includes moieties which function similarly to polynucleotides but which have non-naturally occurring portions. Polynucleotide analogues may have altered sugar moieties, altered base moieties (e.g., inosine, xanthine, hyoxanthine, isocytosine, isoguanine, diaminopurine (DAP), diaminopyrimidine, 2′-deoxyinosine (hypoxanthine deoxynucleotide) derivatives, nitroazole analogues, hydrophobic aromatic non-hydrogen-bonding bases, the thymine analogue 2,4-difluorotoluene, the adenine analogue 4-methylbenzimidazole, isoquinoline, pyrrolo[2,3-b]pyridine, size extended adenine, 2-amino-6-(2-thienyl)purine, pyrrole-2-carbaldehyde), or altered inter-sugar linkages (e.g., phosphoramidate, phosphorothioate, phosphorodithioate, O-methylphosphoroamidite linkages, peptide nucleic acid backbones and linkages, positive backbones; non-ionic backbones, and non-ribose backbones, including those described in U.S. Pat. Nos. 5,235,033 and 5,034,506, which are incorporated by reference). For the purposes of this invention, an polynucleotide analogue having non-phosphodiester bonds, i.e., an altered inter-sugar linkage, can alternately be considered as an “polynucleoside.” Such an polynucleoside thus refers to a plurality of joined nucleoside units joined by linking groups other than native phosphodiester linking groups. Thus polynucleotide of the invention may include a series of nucleosides or nucleoside analogues that are joined together via either natural phosphodiester bonds or via other linkages, including phosphorothioate linkages or the four atom linkers described in, for example, U.S. Pat. No. 5,610,289. Generally, while the linkage is from the 3′ carbon of one nucleoside to the 5′ carbon of a second nucleoside, in some embodiments, the linkage may also include other linkages such as a 2′-5′ linkage.

Polynucleotide or analogues thereof may also comprise other modifications consistent with the spirit of this invention, and in particular such modifications as may enhance cellular uptake, nuclease resistance, and hybridization properties or other useful properties. For example, when the sugar portion of a nucleoside or nucleotide is replaced by a carbocyclic or other moiety, it is no longer a sugar. Moreover, when other substitutions, such a substitution for the inter-sugar phosphorodiester linkage are made, the resulting material is no longer a true nucleic acid species. All such modifications, however, are denominated as polynucleotide analogues or simply polynucleotides. Throughout this specification, reference to the sugar portion of a nucleic acid species shall be understood to refer to either a true sugar or to a species taking the traditional space of the sugar of natural nucleic acids. Moreover, reference to inter-sugar linkages shall be taken to include moieties serving to join the sugar or sugar analogue portions together in the fashion of natural nucleic acids.

The polynucleotide of the invention may be modified at the 5′ end, 3′ end, both 5′ and 3′ ends, and/or at one or more internal nucleotides, or any combinations thereof.

In one embodiment, the polynucleotide of the invention comprises one or more (e.g., about 1, 2, 3, 4, 5, 6, 7, 8, 9, 10, or more) end modifications, which end modifications may be at the 5′ end, the 3′ end, or both ends.

In certain embodiments, the internal nucleotides of the polynucleotide of the invention are modified. As used herein, an “internal” nucleotide is one occurring at any position other than the 5′ end or 3′ end of a nucleic acid molecule, polynucleotide or oligonucleotide. An internal nucleotide can be within a single-stranded molecule or within either strand of a duplex or double-stranded molecule.

In one embodiment, the oligonucleotide of the invention is modified in at least one internal nucleotide, e.g., in at least 2, 3, 4, 5, 6, 7, 8, 9, 10, 11, 12, 13, 14, 15, 16, 17, 18, 19, 20, 21, 22, 23, 24, 25 or more, or all internal nucleotides.

In one embodiment, the polynucleotide of the invention is modified in all nucleotides.

In another embodiment, the polynucleotide of the invention is modified in at least 5%, 10%, 15%, 20%, 25%, 30%, 35%, 40%, 45%, 50%, 55%, 60%, 65%, 70%, 75%, 80%, 85%, 90%, 95% or 100% of the internal nucleotides, or 100% of the nucleotides.

Modifications to the internal or end nucleotides can include, for example, sugar modifications, base modifications, backbone modifications, or combinations thereof.

In one embodiment, the polynucleotide of the invention comprises one or more (e.g., about 1, 2, 3, 4, 5, 6, 7, 8, 9, 10, 11, 12, 13, 14, 15, 16, 17, 18, 19, 20, 21, 22, 23, 24, 25 or more) backbone-modified nucleotides (i.e., modifications to the sugar phosphate backbone). For example, the phosphodiester linkages of natural DNA or RNA may be modified to include at least one of a nitrogen or sulfur heteroatom. In certain backbone-modified polynucleotides, the phosphodiester group connecting adjacent nucleotides may be replaced by a modified group, e.g., a phosphorothioate group.

In one embodiment, the polynucleotide of the invention comprises one or more sugar-modified nucleotides. Sugar-modified nucleotides can include modifications to any substituents of the sugar portion of the nucleotide, e.g., the 2′ moiety of the ribose sugar in a ribonucleotide. The 2′ moiety can be, but is not limited to, H, OR, R, halo, SH, SR, NH₂, NHR, NR₂ or CN, wherein R is C₁-C₆ alkyl, alkenyl, or alkynyl, and halo is F, Cl, Br, or I. In certain embodiments, the modifications are 2′-fluoro, 2′-ammo, and/or 2′-thio modifications. The nucleic acid may comprise a 2′-ribose replacement such as a 2′-O-methyl and 2′-fluoro group, as described in U.S. Pat. No. 7,138,517, the contents of which are incorporated herein by reference. Modified nucleotides also include nucleotides conjugated with cholesterol through a hydroxyprolinol linkage as described in Krutzfeldt et al., Nature, 438:685-689 (2005), Soutschek et al., Nature, 432:173-178 (2004), and U.S. Patent Publication No. 2005/0107325, which are incorporated herein by reference.

In certain embodiments, modifications include uridines or cytidines modified at the 5-position, e.g., 5-(2-amino)propyl uridine, 5-bromo uridine; adenosines and guanosines modified at the 8-position, e.g., 8-bromo guanosine; deaza nucleotides, e.g., 7-deaza-adenosine; 0- and N-alkylated nucleotides, e.g., N6-methyl adenosine are suitable.

In certain embodiments, sugar modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In certain embodiments, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. In addition, 2′-deoxy-nucleotides and 2′-O-Me nucleotides can also be used in the oligonucleotide of the invention. Additional modified residues include, deoxy-abasic, inosine, N3-methyl-uridine, N6,N6-dimethyl-adenosine, pseudouridine, purine ribonucleoside and ribavirin. In one embodiment, one or more 2′ moiety is a methyl group (2′-O-methyl oligonucleotide). In one embodiment, the 2′-O-methyl modified nucleotide occurs on alternating positions (e.g., on the odd or even number nucleotides, over a part or the entire polynucleotide). In one embodiment, the 2′-O-methyl modified nucleotide does not occur on alternating positions (e.g., on the odd or even number nucleotides) over any stretch of 4 or more consecutive nucleotides, or over the entire length of the polynucleotide.

In certain embodiments, the polynucleotide of the invention comprises one or more Locked Nucleic Acids (LNAs). LNAs comprise sugar-modified nucleotides that resist nuclease activities (thus highly stable), and possess single nucleotide discrimination for mRNA (Elmen et al., Nucleic Acids Res., 33(1): 439-447, 2005; Braasch et al., Biochemistry, 42:7967-7975, 2003; Petersen et al., Trends Biotechnol., 21:74-81, 2003). These molecules have 2′-O,4′-C-ethylene-bridged nucleic acids, with possible modifications such as 2′-deoxy-2″-fluorouridme. Moreover, LNAs increase the specificity of polynucleotides by constraining the sugar moiety into the 3′-endo conformation, thereby pre-organizing the nucleotide for base pairing and increasing the melting temperature of the polynucleotide by as much as 10° C. per base.

In certain embodiments, the polynucleotide of the invention comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone, which is highly resistant to nuclease digestion, and imparts improved binding specificity to the molecule (Nielsen et al., Science, 254:1497-1500, 2001).

In certain embodiments, the polynucleotide of the invention comprises Morpholino nucleic acid analog, or “PMO” (phosphorodiamidate morpholino oligo). Morpholinos are synthetic nucleic acid analogs that bind to complementary sequences of RNA by standard nucleic acid base-pairing. Structurally, Morpholinos are similar to DNA in that Morpholinos have standard nucleic acid bases. However, those bases are bound to morpholine rings instead of deoxyribose rings and linked through phosphorodiamidate groups instead of phosphates. Replacement of anionic phosphates with the uncharged phosphorodiamidate groups eliminates ionization in the usual physiological pH range, so Morpholinos in organisms or cells are uncharged molecules. The entire backbone of a Morpholino is made from these modified subunits.

In certain embodiments, the polynucleotide of the invention comprises Glycol Nucleic A (GNA), which is a synthesized polymer similar to DNA or RNA but differing in the composition of its backbone. Specifically, DNA and RNA have a deoxyribose and ribose sugar backbone, respectively, whereas GNA's backbone is composed of repeating glycol units linked by phosphodiester bonds. The glycol unit has just three carbon atoms, yet still shows Watson-Crick base pairing, and the Watson-Crick base pairing is much more stable in GNA than its natural counterparts DNA and RNA as it requires a high temperature to melt a duplex of GNA. The 2,3-dihydroxypropylnucleoside analogues were first prepared by Ueda et al. (1971).

In certain embodiments, the polynucleotide of the invention comprises Threose Nucleic Acid (TNA), which is a synthetic nucleic acid analog similar to DNA or RNA but differing in the composition of its backbone. Specifically, TNA's backbone is composed of repeating threose units linked by phosphodiester bonds. TNA can hybridize with RNA and DNA in a sequence-specific manner. TNA is also capable of Watson-Crick pair bonding, and forming a double helix structure.

In certain embodiments, the polynucleotide of the invention comprises one or more (e.g., about 1, 2, 3, 4, 5, 6, 7, 8, 9, 10, 11, 12, 13, 14, 15, 16, 17, 18, 19, 20, 21, 22, 23, 24, 25 or more) base-modified nucleotides (i.e., the nucleotides contain at least one non-naturally occurring base instead of a naturally occurring base). Bases may be modified to block the activity of adenosine deaminase. Exemplary modified bases include, but are not limited to, uridine and/or cytidine modified at the 5-position (e.g., 5-(2-amino)propyl uridine, 5-fluoro-cytidine, 5-fluoro-uridine, 5-bromo-uridine, 5-iodo-uridine, and 5-methyl-cytidine), adenosine and/or guanosines modified at the 8 position (e.g., 8-bromo guanosine), deaza nucleotides (e.g., 7-deaza-adenosine), and 0- and N-alkylated nucleotides (e.g., N6-methyl adenosine). Base-modified nucleotides for use in the present invention also include, but are not limited to, ribo-thymidine, 2-aminopurine, 2,6-diaminopurine, 4-thio-uridine, and 5-amino-allyl-uridine and the like. It should be noted that the above modifications may be combined.

In certain embodiments, the polynucleotide of the invention, with or without modification, comprises a sequence wherein at least a portion (e.g., the miRNA binding/hybridizing moiety) contains one or more (e.g., about 1, 2, 3, 4, 5, 6, 7, 8, 9, 10, or more) mismatches with the target polynucleotide (e.g., miRNA). In one embodiment, the polynucleotide of the invention, with or without modification, may bind to its target sequence, and may optionally contain one or more mismatches or bulges.

In certain embodiments, the polynucleotide of the invention comprises any combination of two or more (e.g., about 2, 3, 4, 5, 6, 7, 8, 9, 10, or more) modifications as described herein. For example, the polynucleotide of the invention can comprise DNA, RNA, LNA, PNA, or a combination thereof. The polynucleotide of the invention may comprise phosphorothioate linkages throughout, and may additionally comprise one or more sugar-modified nucleotides, such as 2′-fluoro modified ribonucleotides (e.g., 2′-fluoro uridine or 2′-fluoro cytidine), 2′-deoxy ribonucleotides (e.g., 2′-deoxy adenosine or 2′-deoxy guanosine), and/or 2′-O-methyl modified ribonucleotides.

Other modified nucleotides are described in U.S. Pat. No. 5,610,289 (incorporated by reference), which describes therapeutic oligonucleotide analogues having improved nuclease resistance and improved cellular uptake. According to U.S. Pat. No. 5,610,289, replacement of the normal phosphorodiester inter-sugar linkages found in natural oligomers with four atom linking groups forms unique di- and poly-nucleosides and nucleotides useful in therapeutics. More specifically, oligonucleotides or analogues thereof may have at least portions of their backbone linkages modified. In these modifications, the phosphorodiester linkage of the sugar phosphate backbone found in natural nucleic acids has been replaced with various four atom linking groups. Such four atom linking groups maintain a desired four atom spacing between the 3′-carbon of one sugar or sugar analogue and the 4′-carbon of the adjacent sugar or sugar analogue. Oligonucleotide analogues so made are comprised of a selected sequence which is specifically hybridizable with a preselected nucleotide sequence of single stranded or double stranded DNA or RNA. Such oligonucleotides are synthesized conveniently, through known solid state synthetic methodology, to be complementary to or at least to be specifically hybridizable with the preselected target nucleotide sequence of the RNA or DNA. Nucleic acid synthesizers are commercially available and their use is generally understood by persons of ordinary skill in the art as being effective in generating nearly any oligonucleotide or oligonucleotide analogue of reasonable length which may be desired.

According to the invention, the polynucleotide of the invention should be modified as necessary, in part, to improve stability, to prevent degradation in vivo (e.g., by cellular nucleases), to improve cellular uptake, to enhance target efficiency, to improve efficacy in binding (e.g., to the targets), to improve patient tolerance, and/or to reduce toxicity.

In one embodiment, the polynucleotide of the invention has a target binding moiety or portion of about 5-10 nucleotides in length, 10-15 nucleotides in length, 15-20 nucleotides in length, about 20, 21, or 22 nucleotides in length, or about 25-50 nucleotides in length.

In certain embodiments, the targeting moiety or portion is on the 5′ end or the 3′ end of the polynucleotide of the invention, or roughly the middle of the polynucleotide of the invention.

In certain embodiments, the polynucleotide of the invention may be linked to other moieties, such as fluorescent dyes, and may have additional modifications in such other moieties.

The polynucleotide of the invention or portions thereof may be produced enzymatically and/or by partial/total organic synthesis, and any modified nucleotides may be introduced by in vitro enzymatic or organic synthesis.

In one embodiment, the polynucleotide of the invention is prepared chemically. Methods of synthesizing polynucleotide molecules are known in the art, in particular, the chemical synthesis methods as described in, for example, Verma and Eckstein (1998) Annul. Rev. Biochem., 67:99-134.

Alternatively, the polynucleotide of the invention can be prepared by enzymatic transcription from synthetic DNA templates or from DNA plasmids isolated from recombinant host, such as a bacteria. For example, phage RNA polymerases may be used for RNA oligonucleotides, including T7, T3 or SP6 RNA polymerase (Milligan and Uhlenbeck (1989) Methods Enzymol., 180:51-62). The RNA may be dried for storage or dissolved in an aqueous solution. The solution may contain buffers or salts to inhibit annealing, and/or promote stabilization of the single strands.

In another embodiment, polynucleotide of the invention is synthesized directly either in vivo, in situ, or in vitro. An endogenous RNA polymerase in the cell may mediate transcription of the polynucleotide of the invention in vivo or in situ, or a cloned RNA polymerase can be used for transcription of the polynucleotide of the invention in vivo or in vitro. For transcription from a transgene in vivo or an expression construct, a regulatory region (e.g., promoter, enhancer, silencer, splice donor and acceptor, polyadenylation) maybe used to transcribe the polynucleotide of the invention. Inhibition of the target may be targeted by specific transcription in an organ, tissue, or cell type (e.g., endothelial cells or other cells of the blood vessel); stimulation of an environmental condition (e.g., infection, stress, temperature, chemical inducers); and/or engineering transcription at a developmental stage or age.

### 4. Administration and Medical Devices

The “administering” step of the invention may be performed using any medically-accepted means suitable for introducing a therapeutic directly or indirectly into the vasculature of a mammalian subject, including but not limited to injections; oral ingestion; intranasal or topical administration; and the like. In one embodiment, administration of the subject composition comprising the Pdx1 and/or VEGF-A inhibitor compositions is performed intravascularly, such as by intravenous, intra-arterial, or intracoronary arterial injection.

In one embodiment, the subject composition is administered locally, e.g., to the site of angioplasty or bypass. For example, the administering comprises a catheter-mediated transfer of the therapeutic composition into a blood vessel of the mammalian subject, especially into an artery, such as a coronary artery of the mammalian subject. Exemplary materials and methods for local delivery are reviewed in Lincoff et al. (Circulation 90:2070-2084, 1994) and Wilensky et al. (Trends Cardiovasc. Med. 3:163-170, 1993), both incorporated herein by reference.

For example, the composition may be administered using infusion-perfusion balloon catheters, preferably mircroporous balloon catheters, such as those described in the literature for intracoronary drug infusions. See, e.g., U.S. Pat. No. 5,713,860 (Intravascular Catheter with Infusion Array); U.S. Pat. No. 5,087,244; U.S. Pat. No. 5,653,689; and Wolinsky et al. (J. Am. Coll. Cardiol. 15:475-481, 1990) (Wolinsky Infusion Catheter); and Lambert et al. (Coron. Artery Dis. 4:469-475, 1993), all of which are incorporated herein by reference in their entirety. Use of such catheters for site-directed somatic cell gene therapy is described, e.g., in Mazur et al. (Texas Heart Institute Journal 21:104-111, 1994, incorporated herein by reference).

For example, in patients with angina pectoris due to a single or multiple lesions in coronary arteries and for whom PTCA is prescribed on the basis of primary coronary angiogram findings, an exemplary protocol involves performing PTCA through a 7F guiding catheter according to standard clinical practice using the femoral approach. If an optimal result is not achieved with PTCA alone, then an endovascular stent also is implanted. A nonoptimal result is defined as residual stenosis of >30% of the luminal diameter according to a visual estimate, and B or C type dissection. Arterial gene transfer at the site of balloon dilatation is performed immediately after the angioplasty, but before stent implantation, using an infusion-perfusion balloon catheter. The size of the catheter will be selected to match the diameter of the artery as measured from the angiogram, varying, e.g., from 3.0 to 3.5 F in diameter. The balloon is inflated to the optimal pressure.

In another embodiment, intravascular administration with a gel-coated catheter is contemplated, as has been described in the literature to introduce other transgenes. See, e.g., U.S. Pat. No. 5,674,192 (Catheter coated with tenaciously-adhered swellable hydrogel polymer); Riessen et al. (Human Gene Therapy 4:749-758, 1993); and Steg et al. (Circulation 96:408-411, 1997; and Circulation 90:1648-1656, 1994); all incorporated herein by reference.

Briefly, as illustrated in FIG. 1 of US 2005-0256075 A1 (incorporated by reference), a catheter is provided to which an inflatable balloon is attached at a distal end. The balloon includes a swellable hydrogel polymer coating capable of absorbing a solution comprising a subject therapeutic agent. Briefly, DNA in solution (e.g., polynucleotide encoding RNAi reagents against Pdx1 and/or VEGF-A) is applied one or more times ex vivo to the surface of an inflated angioplasty catheter balloon coated with a hydrogel polymer (e.g., Slider with Hydroplus, Mansfield Boston Scientific Corp., Watertown, Mass.). The Hydroplus coating is a hydrophilic polyacrylic acid polymer that is cross-linked to the balloon to form a high molecular weight hydrogel tightly adhered to the balloon. The DNA covered hydrogel is permitted to dry before deflating the balloon. Re-inflation of the balloon intravascularly, during an angioplasty procedure, causes the transfer of the DNA to the vessel wall. Thus, referring again to FIG. 1 of US 2005-0256075 A1, the catheter with attached, coated balloon is inserted into the lumen of a blood vessel while covered by a protective sheath to minimize exposure of the coated balloon to the blood prior to placement at the site of an occlusion. When the instrument has been positioned at the treatment region, the protective sheath is drawn back or the catheter is moved forward to expose the balloon, which is inflated to compress the balloon (and thus the coating) into the vessel wall, causing transfer of the subject therapeutic agent to the tissue, in a manner analogous to squeezing liquid from a compressed sponge or transferring wet paint to a surface by contact.

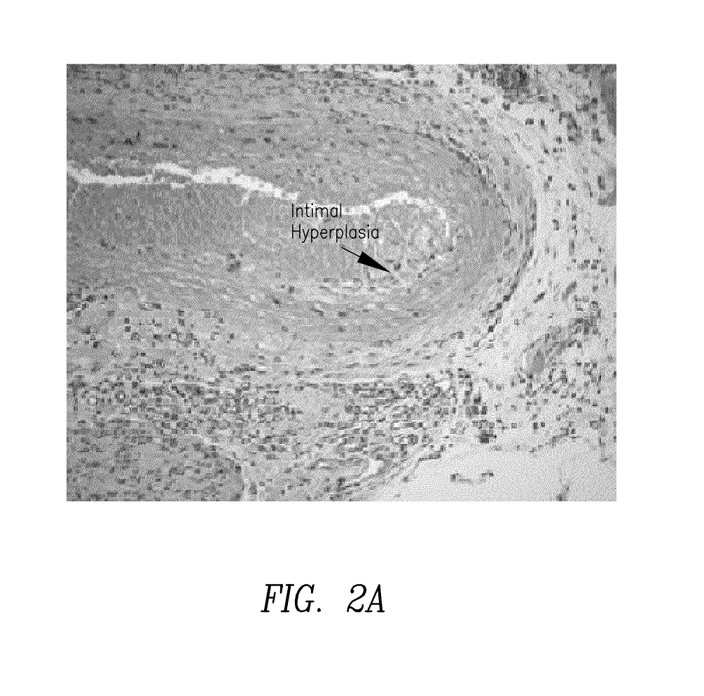

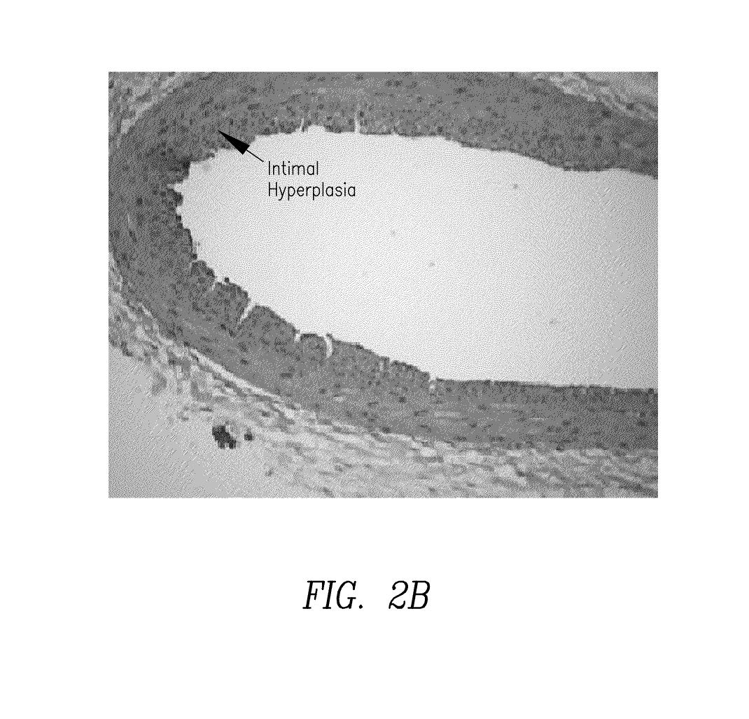

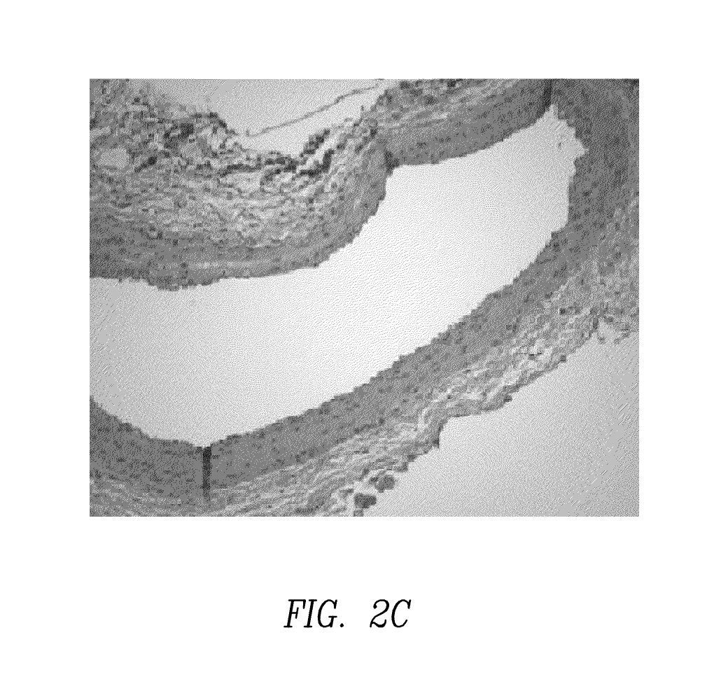

In yet another embodiment, an expandable elastic membrane, film, or similar structure, mounted to or integral with a balloon angioplasty catheter or stent, is employed to deliver the subject therapeutic agent. See, e.g., U.S. Pat. Nos. 5,707,385, 5,697,967, 5,700,286, 5,800,507, and 5,776,184, all incorporated by reference herein. As shown in FIGS. 2A-2B of US 2005-0256075 A1, a single layer or multi-layer, or sheet of elastic membrane material (FIG. 2A of US 2005-0256075 A1) is formed into a tubular structure (FIG. 2B of US 2005-0256075 A1), e.g., by bringing together and adhering opposite edges of the sheet(s), e.g., in an overlapping or a abutting relationship. In this manner the elastomeric material may be wrapped around a catheter balloon or stent. A therapeutic composition is combined with the membrane using any suitable means, including injection molding, coating, diffusion, and absorption techniques. In the multilayer embodiment depicted in those Figures, the edges of the two layers may be joined to form a fluid-tight seal. In a preferred embodiment, one layer of material is first processed by stretching the material and introducing a plurality of microscopic holes or slits. After the layers have been joined together, the sheet can be stretched and injected with the subject therapeutic composition through one of the holes or slits to fill the cavity that exists between the layers. The sheet is then relaxed, causing the holes to close and sealing the therapeutic composition between the layers until such time as the sheet is again stretched. This occurs, for example, at the time that an endovascular stent or balloon covered by the sheet is expanded within the lumen of a stenosed blood vessel. The expanding stent or balloon presses radially outward against the inner surface of the tubular sheet covering, thus stretching the sheet, opening the holes, and delivering the therapeutic agent to the walls of the vessel.

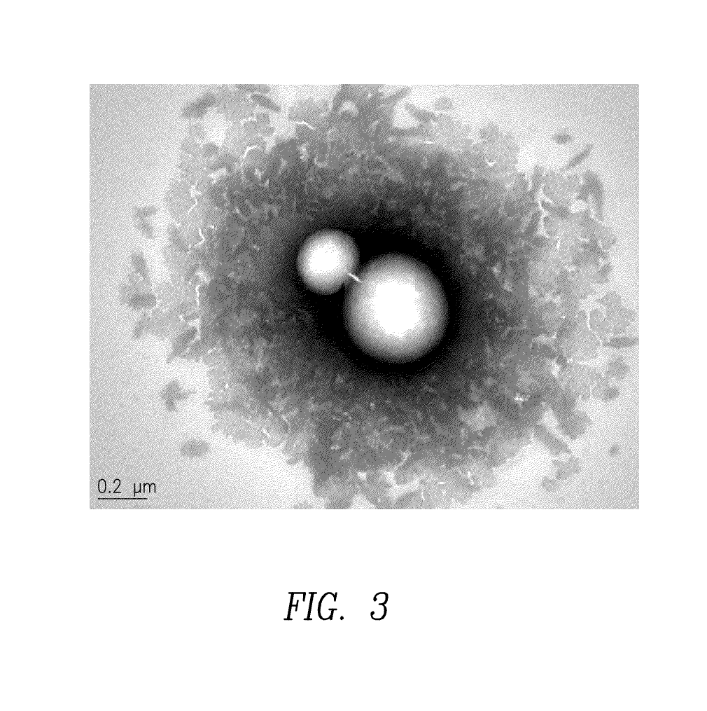

In another variation, the composition containing the subject therapeutic is administered extravascularly, e.g., using a device to surround or encapsulate a portion of vessel. See, e.g., International Patent Publication WO 98/20027, incorporated herein by reference, describing a collar that is placed around the outside of an artery (e.g., during a bypass procedure) to deliver a transgene to the arterial wall via a plasmid or liposome vector. As shown in FIGS. 3A and 3B of US 2005-0256075 A1, an extravascular collar including a void space defined by a wall formed, e.g., of a biodegradable or biocompatible material. The collar touches the outer wall of a blood vessel at the collar's outer extremities. Blood flows through the lumen of the blood vessel. A longitudinal slit in the flexible collar permits the collar to be deformed and placed around the vessel and then sealed using a conventional tissue glue, such as a thrombin glue.

In still another variation, endothelial cells or endothelial progenitor cells are transfected ex vivo with the subject transgene encoding the therapeutic agents, and the transfected cells are then administered to the mammalian subject. Exemplary procedures for seeding a vascular graft with genetically modified endothelial cells are described in U.S. Pat. No. 5,785,965, incorporated herein by reference.

If the mammalian subject is receiving a vascular graft, the subject therapeutic composition may be directly applied to the isolated vessel segment prior to its being grafted in vivo.

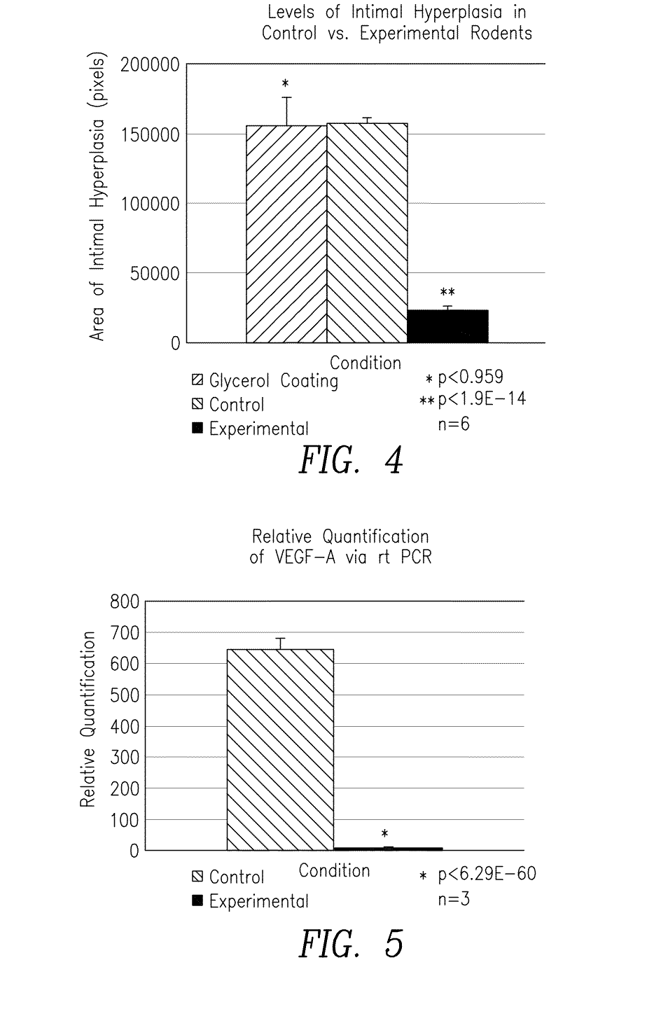

In another preferred embodiment, the administering comprises implanting an intravascular stent in the mammalian subject, where the stent is coated or impregnated with the subject therapeutic gene/protein composition. Exemplary materials for constructing a drug-coated or drug-impregnated stent are described in literature cited above and reviewed in Lincoff et al. (Circulation 90:2070-2084, 1994, incorporated by reference). As shown in FIGS. 4A and 4B of US 2005-0256075 A1, a metal or polymeric wire for forming a stent is coated with a composition such as a porous biocompatible polymer or gel that is impregnated with (or can be dipped in or otherwise easily coated immediately prior to use with) a subject therapeutic composition. The wire is coiled, woven, or otherwise formed into a stent suitable for implantation into the lumen of a vessel using conventional materials and techniques, such as intravascular angioplasty catheterization. Exemplary stents that may be improved in this manner are described and depicted in U.S. Pat. Nos. 5,800,507 and 5,697,967 (Medtronic, Inc., describing an intraluminal stent comprising fibrin and an elutable drug capable of providing a treatment of restenosis); U.S. Pat. No. 5,776,184 (Medtronic, Inc., describing a stent with a porous coating comprising a polymer and a therapeutic substance in a solid or solid/solution with the polymer); U.S. Pat. No. 5,799,384 (Medtronic, Inc., describing a flexible, cylindrical, metal stent having a biocompatible polymeric surface to contact a body lumen); U.S. Pat. Nos. 5,824,048 and 5,679,400; and U.S. Pat. No. 5,779,729; all of which are specifically incorporated herein by reference in the entirety. Implantation of such stents during conventional angioplasty techniques will result in less restenosis than implantation of conventional stents. In this sense, the biocompatibility of the stent is improved.

In another preferred embodiment, the composition comprises microparticles composed of biodegradable polymers such as PGLA, non-degradable polymers, or biological polymers (e.g., starch) which particles encapsulate or are impregnated by the subject polypeptide/polynucleotide. Such particles are delivered to the intravascular wall using, e.g., an infusion angioplasty catheter. Other techniques for achieving locally sustained drug delivery are reviewed in Wilensky et al. (Trends Caridovasc. Med. 3:163-170, 1993, incorporated herein by reference).

Administration via one or more intravenous injections subsequent to the angioplasty or bypass procedure also is contemplated.

The pharmaceutical efficacy of the subject therapeutic agents to prevent stenosis or restenosis of a blood vessel is demonstrated in vivo, e.g., using procedures such as those described in the following examples, actual or prophetic. The examples assist in further describing the invention, but are not intended in any way to limit the scope of the invention.

### EXAMPLES

### Example 1

### Pdx1 Inhibitor (shRNA Against Pdx1) Inhibits IH in a Rat Model of IH

To demonstrate that inhibiting Pdx1 expression inhibits IH in vivo, a construct encoding an anti-Pdx1 shRNA was obtained from Origene (see plasmid map in FIG. 1). The vector encodes an anti-Pdx1 sequence in the form of an shRNA having a 29-bp stem region and a 7-bp loop, which is designed to silence the expression of the Pdx1 gene via RNAi. The plasmid also contains a green fluorescent protein (GFP) coding sequence, which allows easy identification of cells that have taken up the vector. The plasmid was prepared using standard molecular biology protocols.

Next, PLGA nanoparticles were prepared according to Song et al., Colloids and Surfaces A: Physicochemical and Engineering Aspects, 276(1-3):162-167, 2006 (incorporated by reference). Briefly, PLGA 50:50 (100 mg) and Pdx1 shRNA plasmid (500 μL of 400 μg/mL) were used in the preparation of the nanoparticles. Plasmid in TE buffer (500 μL) was poured into the organic phase and sonicated using a probe sonicator at about 10 W for 1 min. to form the primary emulsion. Following sonication, the emulsion was poured into 8 mL of 2% PVA, and was sonicated at 30 W for 3 min. After sonication, the double emulsion was poured into 50 mL of 2% PVA, and solvent was allowed to evaporate at moderate stirring at 1500 rpm in a magnetic stirrer overnight at room temperature. The suspension was further evaporated using a rotovap for 2 hrs at 40° C. in a heated water bath to remove residual solvent. Following evaporation, the suspension was centrifuged at 10,200 rmp for 50 min at 4° C. The particle pellet was resuspended in double-distilled water (20 mL; repeated twice to remove surface bound PVA and plasmid) and lyophilized. After preparation, nanoparticles were analyzed under TEM (transmission electron microscopy) for size and quality. See FIG. 4. The estimated size was roughly between 100-200 nm, as expected. Once the DNA-containing nanoparticles were produced, they were employed during animal surgeries described further below.

All animal procedures were approved by the hospital on which premise the experiment was conducted, and by Institutional Animal Care and Use Committees. Before surgery, balloon catheter and trocar were prepared according to established protocol. A surgical plane of anesthesia was maintained for the duration of the surgery. This level of anesthesia was monitored throughout the surgery, and additional anesthesia was administered as necessary. The experimental rat was placed in the induction chamber to begin anesthesia and isoflurane was then introduced. After roughly 5 minutes, the rat was moved to a heating pad attached to a breathing tube, at which point isoflurane was reduced. The rat was restrained with tape, and the eyes lubricated with ophthalmic solution. Saline was also administered for hydration.

The neck of the rat was then shaved for exposure for surgery. NAIR® brand hair remover was used to remove any remaining fur. This area was then swabbed alternately with betadine and absolute alcohol swabs. At this point, the surgical area was cleansed with 70% alcohol and set up underneath the microscope, with all instruments, suture, cotton-tipped applicators, tape, gauze, etc. properly sterilized and placed on top of the sterile field.

A hole was then cut in the sterile field to allow access to the rat's neck. Specifically, the incision was made with a No. 10 blade scalpel, starting just below the chin of the animal towards the direction of the tail, stopping just above the ribcage at the sternum. Blunt forceps were used to carefully dissect the skin from the underlying glandular tissue. Next, muscle tissues were separated using 3S and 5S forceps, and blunt dissection was performed along the longitudinal left aspect of the central and adjacent muscular tissues (sternocleidomastoid, omohyoid, thyrohyoid, sternohyoid). A retractor was utilized to draw back skin and muscular tissues in order to visualize the left carotid artery and vagus nerve. If arterial branching off the external carotid artery was apparent, the ascending pharyngeal, occipital and/or superior thyroid arteries were completely tied off using short lengths of 6-0 Prolene suture. A 5-6″ 6-0 Prolene suture was tied on the external artery branch as distally and far away from the bifurcation as possible. Distal to the bifurcation of the common carotid artery, a loosely tied ˜3″ 6-0 Prolene blue monofilament suture was looped around the external carotid artery branch. The internal carotid artery was then tied off using a ˜3″ 4-0 Prolene blue monofilament suture. At the most proximal site on the left common carotid artery, as close to the sternum as possible, single ˜3″ suture, size 4-0, Prolene, was placed around the artery. The area was then incubated with 1.5 ccs of lidocaine for several minutes. The left common carotid artery was clamped proximally near the loose suture in order to stop common carotid artery blood flow. The arteriotomy was then made between the two sutures on the external carotid using microscissors. The trocar guiding needle was used to gently insert the un-inflated balloon into the arteriotomy hole. The balloon was then advanced all the way to the arterial clamp on the common carotid artery. The clamp was removed on the common carotid and the catheter was passed all the way down to the aortic arch. The balloon was then inflated and backed out almost all the way to the arteriotomy where it was then deflated. This process of inflation and deflation occurred three times per surgery. After the balloon was fully removed, the clamp was placed back on the proximal common carotid, and the loose suture on the external immediately distal to the arteriotomy was tied off. The clamp and the loose on the common were then removed. After blood flow was reestablished, the suture on the internal was removed as well. Overlying tissues were then replaced and glandular tissues were closed with a 6-0 Prolene running stitch. The skin of the animal was then closed with interrupted sutures using 6-0 Prolene.

Once the animal was sutured, ketorolac, an analgesic, was immediately provided and anesthesia halted. The rat was then cleaned using gauze and sterile water and then placed on a heating pad and monitored to ensure comfort. Once the rat had displayed signs of alertness and was ambulatory, it was returned to its cage and given sufficient food and drink. Instruments were then thoroughly cleaned and sterilized. The surgical area was also swabbed with 70% alcohol and disinfected. Finally, each animal was given daily assessments for two weeks following the surgery to ensure normal and healthy recovery.

In total, nine surgeries were performed on nine separate rats. The initial two surgeries represented the first control group—standard left carotid artery injury surgeries. The next two surgeries comprised the second control group—rats which received a standard left carotid artery injury surgery, modified slightly to include a glycerol coating without nanoparticles-carried plasmids on the catheter to test whether or not the substance (without the shRNA-encoding plasmid) had any noteworthy effects on the artery and/or the overall health of the animal before the nanoparticle treatment was delivered. The balloon was immersed in glycerol until 100 μL had covered its surface. Another two surgeries served as an experimental group to compare the method of delivery with the glycerol-coated balloon to a more direct method of delivery: infusion. After the arteriotomy was performed, the artery was cannulated (filled) with a syringe of 100 μL of 50% glycerol and flushed. The procedure then proceeded as previously described. The last three surgeries served as the experimental group utilizing the nanoparticles suspended in the glycerol that coated the balloon.

After two weeks, each rat was euthanized via perfusion fixation to allow for tissue harvesting of the artery. The tissues were then subjected to tri-chrome stain, and the tri-chrome stained slides were analyzed via Image-J, an NIH software which determines the amount of intimal hyperplasia. Finally, the rest of the tissue harvested was dissolved in TRIZOL (Life Technologies) to dissolve the artery into a stable form to preserve RNA for harvesting. Once the RNA was harvested via a standard isolation protocol involving chloroform for phase separation, isopropyl alcohol for RNA precipitation, and ethanol for RNA wash, the RNA was subsequently analyzed via rt-PCR.

Concerning tri-chrome stain, once the arteries were perfused from the rats, they were either embedded in a non-stained paraffin or processed in a standard procedure for trichrome staining, which clearly stains the three layers of the artery to define the intima, the media, and the adventitia. The stained arteries suggest that the intima of the experimental condition expressed essentially no regrowth, whereas the standard control arteries with no glycerol coating on the balloon, as expected, had significant neo-intima formation (p<2.7E-8), as did the control with the glycerol coating (p<1.9E-14). The intimal regrowth of the various conditions is visibly highlighted in FIGS. 2A-2C.

Intimal hyperplasia was further quantified using a software program, Image J, developed by the National Institutes of Health, which calculates the intimal area through image analysis. The various intimal hyperplasia levels were graphed against the different conditions, and are depicted in FIG. 4. Rats that received gene delivery via infusion developed severe blood clotting, which would eventually completely clog the artery. Thus, this avenue was not an option for inhibiting intimal hyperplasia, as it created even more serious issues.

Next, gene delivery was confirmed using fluorescence microscopy, based on the fact that the shRNA-encoding plasmid also encodes the marker GFP. Under the UV light, gene delivery was confirmed as the intimal cells of the experimental condition did, in fact, fluoresce (results not shown).

Finally, reverse transcriptase polymerase chain reaction was performed on cDNA extracted from rat arteries. FIG. 5 shows gene expression level for VEGF-A as measured by RT-PCT. It was found that the experimental condition resulted in about 68-fold lower levels of VEGF-A expression than the glycerol-control, a statistically significant difference with a p-value of 6.29e-60.

Overall, the tri-chrome stain result revealed that, under the experimental condition, rats receiving the nanoparticle-mediated gene therapy (e.g., shRNA mediated knockdown of Pdx1 expression) suffered appreciably less intimal re-growth. This strongly suggests that these experimental rats did not develop intimal hyperplasia to the same degree that the control rats did. These data demonstrated that delivery of the anti-Pdx1 shRNA coding sequence to the vessel via nanoparticles is a viable method of reducing the degree of intimal hyperplasia in vivo. This procedure ensures the opening of the blood vessel for adequate blood flow following cardiovascular intervention.

The GFP data showed that the Pdx1 vector was indeed delivered to the blood vessel and the encoded genes were properly expressed in the exact location intended. More importantly, the result demonstrates that nanoparticle-mediated surgical delivery of therapeutic agents (e.g., genes or coding sequences) can be used to deliver other gene-based therapies, thus representing a new, more efficient, and more specific gene-delivery system for arterial and venous genes.

The rt-PCR result also clarifies the relationship between Pdx1 and VEGF-A. While not wishing to be bound by any particular theory, Pdx1 is required for VEGF-A expression at least in IH tissues, and inhibiting VEGF-A expression by inhibiting Pdx-1 expression significantly (p<6.29e-60) reduced neo-intima materialization, as confirmed by tri-chrome staining.

The significance of this finding is highlighted by the elucidation of a new signaling pathway and its role in regulating intimal hyperplasia. This connection also provides a new role of Pdx1 in the multiple diseases and conditions associated with the VEGF-A gene.

The data further clarifies the impact of glycerol infusion. As previously discussed, pure glycerol infused as compared to pure glycerol coated on a balloon results in blood clot formation. This suggests that infusions of glycerol should not be used in arteries irrespective of the disease being treated. The only way this treatment differed from the pure glycerol coated on a balloon was in the method of distribution, further suggesting that while glycerol can be used for medical treatments in the body, the route of administration must be carefully chosen.

The new method of gene delivery provides the ability to treat a multitude of both arterial and venous diseases. The encapsulation of shRNA and other therapeutic agents via nanoparticles can be extended to any and all blood vessel diseases (such as coronary artery disease, the leading cause of death in the United States), and a surgical delivery can be employed in many cardiovascular surgeries.

### Example 2

### Use of Adenovirus-Mediated Pdx1 Inhibition to Prevent Restenosis

This example relates to an in vivo rabbit restenosis model for demonstrating the efficacy of adenovirus-mediated intravascular Pdx1 inhibitor (shRNA) gene transfer for the prevention of post-angioplasty restenosis.

### A. Materials and Methods

### 1. Adenoviral Constructs

An adenovirus plasmid containing a cDNA encoding a Pdx1 inhibitor (e.g., shRNA against Pdx1) operably linked to a cytomegalovirus (CMV) promoter and human growth hormone polyadenylation signal sequence is constructed as follows. A DNA fragment comprising a CMV promoter sequence is prepared by digesting the pcDNA3.1+ vector (Invitrogen) with Sal I and filling-in the 5′ overhangs with the Klenow enzyme. The CMV promoter (nucleotides 5431-911) is excised from the vector with Hind III and isolated. An shRNA designed to silence human Pdx1 is prepared by PCR with flanking RE sites for cloning into the vector. A human growth hormone polyadenylation signal (˜860 bp) is excised from an αMHC vector with SalI and BamHI. The CMV promoter, Pdx1 shRNA coding sequence, and hGH polyadenylation signal fragments are simultaneously ligated into a BamHI and EcoRV-digested pCRII vector. The resulting construct is opened with B glII and partially-digested with BamHI. The full transcriptional unit is ligated into BglII-opened pAdBglII vector. This construct is then used to create recombinant adenovirus containing the CMV-Pdx1 shRNA-hGH transcriptional unit, using standard homologous recombination techniques (Barr et al., Gene Ther. 1:51-58, 1994). Replication-deficient E1-E3 deleted adenoviruses are produced in 293 cells and concentrated by ultracentrifugation using techniques known in the literature. A control plasmid comprising the lacZ gene operably linked to the same promoter is also used (Laitinen et al., Hum. Gene Ther. 9:1481-1486, 1998). The lacZ adenovirus has a nuclear targeted signal, to direct the β-galactosidase expression to the nucleus. Replication-deficient E1-E3 deleted adenoviruses are produced in 293 cells and concentrated by ultracentrifugation (Barr et al., 1994). The adenoviral preparations are analyzed for the absence of helper viruses and bacteriological contaminants.

### 2. Animal Model

New Zealand White rabbits are employed for the gene transfer study. A first group of rabbits is fed a 0.25% cholesterol diet for two weeks, then subjected to balloon denudation of the aorta, then subjected three days later to the adenovirus-mediated gene transfer. A second group of rabbits is only subjected to the gene transfer. Animals are sacrificed 2 or 4 weeks after the gene transfer. The number of experimental (e.g., Pdx1 shRNA) and control (lacZ) animals in both study groups is 6.

In the first group of rabbits, the whole aorta, beginning from the tip of the arch, is denuded using a 4.0 F arterial embolectomy catheter (Sorin Biomedical, Irvine, Calif.). The catheter is introduced via the right iliac artery up to the aortic arch and inflated, and the aorta is denuded twice.

### 3. Gene Transfer

The gene transfer is performed using a 3.0 F channel balloon local drug delivery catheter (Boston Scientific Corp., Maple Grove, Mass.). Using fluoroscopical control, the balloon catheter is positioned caudal to the left renal artery, in a segment free of side branches, via a 5 F percutaneous introducer sheath (Arrow International, Reading, Pa.) in the right carotid artery and inflated to 6 ATM with a mixture of contrast media and saline. The anatomical location of the balloon catheter is determined by measuring its distance from the aortic orifice of the left renal artery. Virus titer of 1.15×10¹⁰ plaque forming units (pfu) is administered to each animal in a final volume of 2 mL (0.9% NaCl), and the gene transfer is performed at 6 ATM pressure for 10 minutes (0.2 mL/min). In the second study group the animals have only gene transfer and they are sacrificed 2 weeks after the gene transfer. The number of animals in each study group (0.9% NaCl only; lacZ gene transfer; and Pdx1 shRNA gene transfer) is 3. All studies are approved by Experimental Animal Committee or other similar appropriate authority.

### 4. Histology

Three hours before sacrifice, the animals are injected intravenously with 50 mg of BrdU dissolved in 40% ethanol. After the sacrifice, the aortic segment where the gene transfer have been performed is removed, flushed gently with saline, and divided into five equal segments. The proximal segment is snap frozen in liquid nitrogen and stored at −70° C. The next segment is immersion-fixed in 4% paraformaldehyde/15% sucrose (pH 7.4) for 4 hours, rinsed in 15% sucrose (pH 7.4) overnight, and embedded in paraffin. The medial segment is immersion-fixed in 4% paraformaldehyde/phosphate buffered saline (PBS) (pH 7.4) for 10 minutes, rinsed 2 hours in PBS, embedded in OCT compound (Miles), and stored at −70° C. The fourth segment is immersion-fixed in 70% ethanol overnight and embedded in paraffin. The distal segment is directly stained for β-galactosidase activity in X-GAL staining solution at +37° C. for 16 hours, immersion-fixed in 4% paraformaldehyde/15% sucrose (pH 7.4) for 4 hours, rinsed in 15% sucrose overnight, and embedded in paraffin. Paraffin sections are used for immunocytochemical detection of smooth muscle cells (SMC), macrophages, and endothelium. Gene transfer efficiency is evaluated using X-GAL staining of OCT-embedded tissues. BrdU-positive cells are detected according to manufacturer's instructions. Morphometry is performed using haematoxylin-eosin stained paraffin sections using image analysis software. Measurements are taken independently by two observers from multiple sections, without knowledge of the origin of the sections. Intima/media (I/M) ratio is used as a parameter for intimal thickening.

### Example 3

### Use of Naked Pdx1 shRNA Transgene Therapy to Prevent Restenosis

The procedures described in Example 1 or 2 are repeated, with the following modifications. Instead of using the constructs above, such as a plasmid or an adenovirus vector, for delivery of the Pdx1 shRNA transgene, a mammalian expression vector is constructed for direct gene transfer (of naked plasmid DNA). The Pdx1 shRNA coding sequence is operably linked to a suitable promoter, such as the CMV promoter, and preferably linked to a suitable polyadenylation sequence, such as the human growth hormone polyadenylation sequence. Exemplary Pdx1 shRNA vectors can be modeled from vectors that have been described in the literature to perform vector-free gene transfer for other growth factors. See, e.g., Isner et al., Circulation 91:2687-2692 (1995); and Isner et al., Human Gene Therapy 7:989-1011, 1996, both incorporated herein by reference. A similar construct comprising a lacZ gene is used as a control.

A Hydrogel-coated balloon catheter (Boston Scientific) is used to deliver the Pdx1 shRNA transgene essentially as described in Asahara et al., Circulation 94:3291-3302 (Dec. 15, 1996), incorporated herein by reference. Briefly, an angioplasty balloon is prepared ex vivo by advancing the deflated balloon completely through a teflon protective sheath (Boston Scientific). The balloon is inflated and a conventional pipette is used to apply the transgene construct (e.g., 50-5000 μg transgene DNA in a saline solution) to the Hydrogel polymer coating the external surface of the inflated balloon. After the transgene solution has dried, the balloon is deflated, withdrawn into the protective sheath, and re-inflated to minimize blood flow across the balloon surface until the balloon is properly positioned in the target artery.

Intima/media (I/M) ratio is again used as a parameter for intimal thickening. Reduced I/M ratio in animals treated with the Pdx1 shRNA transgene-coated balloon catheter is considered indicative of therapeutic efficacy. As described in Example 2, comparison of the therapeutic efficacy of Pdx1 shRNA gene transfer with other therapies can be conducted in parallel.

### Example 4

### Use of Pdx1 shRNA Gene Therapy to Prevent Restenosis Following Angioplasty with Stent

The procedures described in the preceding examples are repeated with the modification that initial balloon angioplasty is accompanied by implantation of a coronary stent using conventional procedures. The Pdx1 shRNA transgene is delivered concurrently or immediately before or after stent implantation essentially as described in the preceding examples. Increased quantities (e.g., two-fold to ten-fold) of the transgene (compared to angioplasty without stent) and increased transfection time may be desirable, as described in Van Belle et al., J. Am. Coll. Cardiol. 29:1371-1379, 1997, incorporated by reference herein. Decreased neointimal thickening and/or decreased thrombotic occlusion in the Pdx1 shRNA-treated animals versus control animals treated with a marker gene is considered evidence of the efficacy of the Pdx1 shRNA gene therapy.

### Example 5

### Use of an Extravascular Collar to Reduce Vascular Stenosis

An inert silicone collar such as described in International Patent Publication No. WO 98/20027 is surgically implanted around the carotid arteries of New Zealand White Rabbits. The collar acts as an irritation agent that will induce intimal thickening, and contains a reservoir suitable for local delivery of a Pdx1 shRNA transgene or protein pharmaceutical formulation. Gene transfer, using the Pdx1 shRNA adenovirus construct or control construct described in Example 2 is initiated five days later by injecting 10⁸-10¹¹ pfu into the collar. Animals are sacrificed 14 or 28 days later and histological examinations are performed as described in Example 2. Intima/media thickness ratio (Yla-Herttuala et al., Arteriosclerosis, 6: 230-236, 1986) is used as an indicia of stenosis. Reduced I/M ratio in the Pdx1 shRNA-transfected rabbits, as compared to the lacZ control rabbits, indicates therapeutic efficacy of Pdx1 shRNA gene transfer for preventing arterial stenosis.

### Example 6

### Use of VEGF-C Polypeptides to Reduce or Prevent Restenosis

The procedures described in Example 2 are repeated except, instead of treating the test animals with an adenovirus containing a Pdx1 shRNA transgene or lacZ control, the animals are treated with a composition comprising a Pdx1 antibody as a Pdx1 inhibitor in a pharmaceutically acceptable carrier (e.g., isotonic saline with serum albumin), or with carrier solution alone as a control. Test animals receive either 10, 100, 250, 500, 1000, or 5000 μg of a Pdx1 antibody via intra-arterial infusion, e.g., as described in Example 2. A second group of animals additionally receive an injection of the Pdx1 antibody 7 days later. The animals are sacrificed and histological examination performed as described in Example 2. Reduced I/M ratio in the Pdx1 antibody-treated animals versus control animals provides evidence of the therapeutic efficacy of Pdx1 antibody treatment. Repetition of the experiment using various sustained-release Pdx1 antibody formulations and materials as described above is expected to further enhance the therapeutic efficacy of the Pdx1 antibody. Moreover, a treatment regimen comprising the simultaneous administration of Pdx1 antibody (to provide immediate therapy to the target vessel) with a Pdx1 shRNA transgene (to provide sustained therapy for several days or weeks) is specifically contemplated as a variation of the invention.

### Example 7

### Anti-Stenosis/Anti-Restenosis Activity of VEGF-A Inhibitor

The procedures described in the preceding examples are repeated using a composition comprising a VEGF-A inhibitor (e.g., shRNA) in lieu of the Pdx1 shRNA, to demonstrate the ability of VEGF-A inhibitor to prevent stenosis or restenosis of a blood vessel.

While the present invention has been described in terms of specific embodiments, it is understood that variations and modifications will occur to those in the art, all of which are intended as aspects of the present invention. Accordingly, only such limitations as appear in the claims should be placed on the invention.

### REFERENCES

1. Coronary Artery Bypass Grafting (CABG)—University of Michigan Cardiac Surgery. (n.d.). University of Michigan Health System. Retrieved Sep. 25, 2013, from www dot med dot umich dot edu slash cardiac-surgery slash patient slash adult slash adultcandt slash cabg dot shtml.
2. Spencer, F. C., Isom, O. W., Classman, E., Boyd, A. D., Engelman, R. M., & Reed, G. E. (1974). The Long-Term Influence of Coronary Bypass Grafts On Myocardial Infarction And Survival. Annals of Surgery, 180(4), 439-451.
3. “Miracle stents”—a future without restenosis. (n.d.). National Center for Biotechnology Information. Retrieved Sep. 25, 2013, from www dot ncbi dot nlm dot nih dot gov slash pmc slash articles slash PMC2323487.
4. “Intimal hyperplasia I Radiology Reference Article I Radiopaedia.org.” Collaborative Radiology resource. N.p., n.d. Web. 26 Sep. 2013. <radiopaedia dot org slash articles slash intimal-hyperplasia>.
5. The Role of Vascular Endothelial Growth Factor in Restenosis. (n.d.). Circulation. Retrieved Sep. 25, 2013, from circ dot ahajournals dot org slash content slash 110 slash 16 slash 2283 dot full.
6. PDX1 Gene—GeneCards|PDX1 Protein|PDX1 Antibody. (n.d.). GeneCards—Human Genes|Gene Database|Gene Search. Retrieved Sep. 25, 2013, from www dot genecards dot org slash cgi-bin slash carddisp dot pl?gene=PDX1.
7. The use of DNA viruses as vectors for gene therapy. [Gene Ther. 1994]—PubMed—NCBI. (n.d.). National Center for Biotechnology Information. Retrieved Sep. 25, 2013, from www dot ncbi dot nlm dot nih dot gov slash pubmed slash 7584103.
8. Wagner, D. E., & Bhaduri, S. B. (2011). Progress And Outlook Of Inorganic Nanoparticles For Delivery Of Nucleic Acid Sequences Related To Orthopedic Pathologies: A Review. Tissue Engineering Part B: Reviews.
9. Qiagen Maxiprep Protocol. (n.d.). University of California. Retrieved Sep. 25, 2013, from www dot mcdb dot ucla dot edu slash Research slash Banerjee slash protocols slash maxiprep—Qiagen.pdf.
10. Song, K., Lee, H., Choung, I., Cho, K., Ahn, Y., & Choi, E. (2006). The Effect Of Type Of Organic Phase Solvents On The Particle Size Of Poly(d,l-lactide-co-glycolide) Nanoparticles. Colloids and Surfaces A: Physicochemical and Engineering Aspects, 276(1-3), 162-167.
11. Rat carotid artery balloon injury model. [Methods Mol Med. 2007]—PubMed—NCBI. (n.d.). National Center for Biotechnology Information. Retrieved Sep. 25, 2013, from www dot ncbi dot nlm dot nih dot gov slash pubmed slash 18287662.
12. Perfusion fixation protocol. (n.d.). Antibodies, Proteins, Kits and Reagents for Life Science|Abcam. Retrieved Sep. 25, 2013, from www dot abcam dot com slash index dot html?pageconfig=resource&rid=11466.
13. OriGene—RNAi—HuSH Vector. (n.d.). OriGene—Your Gene Company—Over 37,000 Full Length ORF cDNA Clones. Retrieved Sep. 25, 2013, from www dot origene dot com slash shRNA slash vector_information.aspx#pGFP-V-RS.

All references cited herein above are incorporated herein by reference.

What is claimed is:

1. A medical device comprising a surface designed to contact the lumen of a blood vessel in a mammal, wherein the surface is coated by a composition comprising a VEGF-A inhibitor and/or a Pdx1 inhibitor, and wherein said VEGF-A inhibitor inhibits expression of VEGF-A, and said Pdx1 inhibitor inhibits expression of Pdx1.

2. The medical device of claim 1, wherein the composition further comprises glycerol.

3. The medical device of claim 1, which is a stent (e.g., an intravascular stent), a catheter (e.g., an intravascular catheter, a balloon catheter), an angioplastic balloon, an extravascular collar, an elastomeric membrane adapted to cover a surface of an intravascular stent or catheter, or a combination thereof.

4. A method of treating or preventing stenosis, restenosis, or intimal hyperplasia (IH) in a mammal in need of treatment or prevention, the method comprising administering a therapeutically or prophylactically effective amount of a Pdx1 inhibitor and/or a VEGF-A inhibitor to the mammal in need thereof.

5. The method of claim 4, wherein the Pdx1 inhibitor and/or the VEGF-A inhibitor is administered by contacting the lumen of a blood vessel in the mammal afflicted with stenosis, restenosis, or intimal hyperplasia (IH) with a surface of a medical device, wherein the surface is coated by a composition comprising the VEGF-A inhibitor and/or the Pdx1 inhibitor.

6. The method of claim 5, wherein the mammal is a human, or a rodent (e.g., a rat).

7. The method of claim 6, wherein the VEGF-A inhibitor and/or the Pdx1 inhibitor is administered prophylactically to the blood vessel shortly before, concurrently with, or shortly after an angioplasty procedure, or a procedure to perform a vascular graft.

8. The method of claim 7, wherein the VEGF-A inhibitor and/or the Pdx1 inhibitor is administered with a device employed in the angioplasty selected from the group consisting of a catheter, a stent, an expandable elastic membrane, and a combination thereof.

9. The method of claim 7, wherein the VEGF-A inhibitor and/or the Pdx1 inhibitor is administered with a device used in a vascular graft procedure (e.g., an extravascular collar).

10. The method of claim 5, wherein the blood vessel is an artery (or a vein).

11. The method of claim 5, wherein the Pdx1 inhibitor and/or the VEGF-A inhibitor is a polynucleotide.

12. The method of claim 11, wherein the polynucleotide inhibits Pdx1 expression and/or VEGF-A expression via RNA interference (RNAi).

13. The method of claim 12, wherein the polynucleotide is an shRNA (short hairpin RNA), a dsRNA that can be processed by an RNAse III into siRNA, or an miRNA or precursor thereof.

14. The method of claim 13, wherein the polynucleotide comprises a modified sugar moiety (e.g., 2-O-Me), a modified base moiety (e.g., nebularine or xanthosine nucleotide), a modified inter-sugar linkage (e.g., phosphorothioate), or combinations thereof.

15. The method of claim 13, wherein the polynucleotide comprises a locked nucleic acid (LNA™), a deoxyribonucleic acid (DNA), a ribonucleic acid (RNA), or a combination thereof.

16. The method of claim 11, wherein the polynucleotide encodes a product that inhibits Pdx1 expression and/or VEGF-A expression via RNA interference (RNAi).

17. The method of claim 16, wherein the product is an shRNA (short hairpin RNA), a dsRNA that can be processed by an RNAse III into siRNA, or an miRNA or precursor thereof.

18. The method of claim 16, wherein the product is expressed from an operably linked promoter on the polynucleotide.

19. The method of claim 16, wherein expression of the product in endothelial cells of the blood vessel contacted by the surface leads to reduced expression of Pdx1 and/or VEGF-A in said endothelial cells.

20. The method of claim 16, wherein expression of the product in endothelial cells of the blood vessel contacted by the surface leads to inhibition of stenosis, restenosis, or IH of the blood vessel.

21. The method of claim 11, wherein the polynucleotide is a plasmid vector (e.g., naked DNA plasmid vector), or a viral vector (e.g., adenoviral vector preferably a replication-deficient adenoviral vector, AAV vector, retroviral vector, lentiviral vector, lipofectin-mediated gene transfer vector, liposome).

22. The method of claim 4, further comprising identifying the mammal in need of treatment as being a candidate for administering the Pdx1 inhibitor and/or the VEGF-A inhibitor.

23. The method of claim 22, wherein the mammal has been treated for a stenosed blood vessel, has a stenosed blood vessel, or will be treated for a stenosed blood vessel.

24. The method of claim 5, wherein the blood vessel is a grafted blood vessel.


SEQUENCE LISTING (2 sequences)

SEQ ID NO: 1 - DNA, 2573 bp, Homo sapiens
gggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaacctttctggcagtgggatgattcgaattcacttttaaaattaaattagcgtgttttgttttg

SEQ ID NO: 2 - DNA, 57 bp, Artificial Sequence
  Oligonucleotide
nnnnnnnnnnnnnnnnnnnntcaagagnnnnnnnnnnnnnnnnnnnnttttttggaa